(19) United States
(12) Patent Application Publication
(10) Pub. No.: US 2005/0196864 A1
(43) Pub. Date: Sep. 8, 2005

(54) Induction and high-yield preparative purification of mesencephalic dopaminergic neuronal progenitor cells and dopaminergic neurons from human embryonic stem cells

(76) Inventors: Steven A. Goldman, Webster, NY (US); Neeta Singh Roy, New York, NY (US); Takahiro Nakano, Salt Lake City, UT (US)

Correspondence Address: Michael L. Goldman;Nixon Peabody LLP, Clinton Square, P.O. Box 31051, Rochester, NY, 14603-1051, US

(21) Appl. No.: 11/055,506

(22) Filed: Feb. 10, 2005

Related U.S. Application Data
(60) Provisional application No. 60/543,189, filed on Feb. 10, 2004.

(51) Int. Cl. C12N 15/86; C12N 15/87; C12N 5/08

(52) U.S. Cl. 435/456; 435/368; 435/459

(57) ABSTRACT

The present invention relates to an enriched or purified population of dopaminergic neuronal progenitor cells and an enriched or purified population of dopaminergic neurons. These enriched or purified populations are derived from a population of embryonic stem cells by inducing production of dopaminergic neuronal progenitor cells. A promoter or enhancer which functions only in dopaminergic neuronal progenitor cells is selected and a nucleic acid molecule encoding a marker protein under control of said promoter or enhancer is introduced into the induced population of embryonic stem cells. The dopaminergic neuronal progenitor cells are allowed to express the marker protein, and the cells expressing the marker protein are separated from the induced population of embryonic stem cells. As a result, an enriched or purified population of dopaminergic neuronal progenitor cells is isolated. Alternatively, the nucleic acid molecule encoding the marker protein under control of the promoter or enhancer is introduced into the population of human embryonic stem cells followed by induction of the population of embryonic stem cells.

This application claims benefit of U.S. Provisional Patent Application Ser. No. 60/543,189, filed Feb. 10, 2004.

The subject matter of the application was made with support from the United States Government under National Institutes of Health Grant No. RO1NS33106. The U.S. government may have certain rights.

### FIELD OF THE INVENTION

The present invention relates to an enriched or purified population of dopaminergic neuronal progenitor cells, an enriched or purified population of dopaminergic neurons, as well as a method of producing such enriched or purified populations from a population of embryonic stem cells.

### BACKGROUND OF THE INVENTION

Human embryonic stem (“ES”) cells have been shown to generate the entire range of major somatic cell lineages (Reubinoff et al., “Embryonic Stem Cell Lines From Human Blastocysts: Somatic Differentiation In Vitro,” Nat Biotechnol 18:399-404 (2000); Shamblott et al., “Derivation of Pluripotent Stem Cells From Cultured Human Primordial Germ Cells,” Proc Natl Acad Sci USA 95:13726-13731 (1998); Thomson et al., “Embryonic Stem Cell Lines Derived From Human Blastocysts,” Science 282:1145-1147 (1998)), including neurons and glia (Reubinoff et al., “Neural Progenitors From Human Embryonic Stem Cells,” Nat Biotechnol 19:1134-1140 (2001); Zhang et al., “In Vitro Differentiation of Transplantable Neural Precursors From Human Embryonic Stem Cells,” Nat Biotechnol 19:1129-1133 (2001)). Neural stem cells may be generated from human embryonic stem (“hES”) cells by the action of FGF2 alone, an inductive step so minimal as to suggest that neurogenesis may be a default pathway for hES differentiation. From these, a variety of specific neuronal phenotypes have been induced in ES cell cultures; importantly, the rules for generating given lineages have thus far appeared relatively conserved between murine and human ES cells. Dopaminergic neurons, expressing transcription factors typical of midbrain, have been selectively induced from both murine and human ES cells, under the combined influence of SHH and FGF8 (Lee et al., “Efficient Generation of Midbrain and Hindbrain Neurons From Mouse Embryonic Stem Cells,” Nature Biotechnol 18:675-679 (2000); Perrier et al., “Derivation of Midbrain Dopamine Neurons From Human Embryonic Stem Cells,” Proc Natl Acad Sci 101:12543-12548 (2004)). However, although SHH/FGF8 induction strongly potentiated dopaminergic neurogenesis by these cells, it has proven insufficient to achieve high levels of enrichment of dopaminergic neurons. Co-culture with a variety of stromal feeder cells and striatal astrocytes have been used to accentuate dopaminergic neuronal production in vitro (Buytaert-Hoefen et al., “Generation of Tyrosine Hydroxylase Positive Neurions From Human Embryonic Stem Cells After Coculture With Cellular Substrates and Exposure to GDNF,” Stem Cells 22:669-674 (2004); Kawasaki et al., “Induction of Midbrain Dopaminergic Neurons From ES Cells by Stromal Cell-derived Inducing Activity,” Neuron 28:31-40 (2000); Perrier et al., “Derivation of Midbrain Dopamine Neurons From Human Embryonic Stem Cells,” Proc Natl Acad Sci USA 101:12543-12548 (2004)). Yet, despite strong evidence for the existence of mesencephalic glial signals for dopaminergic neuronal differentiation and survival (Takeshima et al., “Mesencephalic Type 1 Astrocytes Rescue Dopaminergic Neurons From Death Induced by Serum Deprivation,” J Neurosci 14:4769-4779 (1994); Wagner et al., “Induction of a Midbrain Dopaminergic Phenotype in Nurr1-Overexpressing Neural Stem Cells by type 1 Astrocytes,” Nature Biotechnology 17:653-659 (1999)), no studies to date have attempted to mimic developmental dopaminergic induction from ES cells with mesencephalic glia. This has in part been due to the scarcity of native human fetal mesencephalic glia, and the lack of available lines of these cells.

The acquisition of highly enriched dopaminergic populations is a particularly important prerequisite to using hES-derived dopaminergic neurons for cell-based therapy, since non-dopaminergic neuronal derivatives may yield unpredictable or deleterious neurological side-effects, while incompletely differentiated hES cells are potentially tumorigenic upon implantation (Bjorklund et al., “Embryonic Stem Cells Develop Into Functional Dopaminergic Neurons After Transplantation in a Parkinson Rat Model,” Proc Natl Acad Sci USA 99:2344-2349 (2002)).

The present invention is directed to overcoming this need in the art.

### SUMMARY OF THE INVENTION

The present invention relates to a method of isolating an enriched or purified population of dopaminergic neuronal progenitor cells from a population of embryonic stem cells. This involves providing a population of embryonic stem cells and inducing production of dopaminergic neuronal progenitor cells from the population of embryonic stem cells. A promoter or enhancer which functions only in dopaminergic neuronal progenitor cells is selected and a nucleic acid molecule encoding a marker protein under control of said promoter or enhancer is introduced into the induced population of embryonic stem cells. The dopaminergic neuronal progenitor cells are allowed to express the marker protein, and the cells expressing the marker protein are separated from the induced population of embryonic stem cells. As a result, an enriched or purified population of dopaminergic neuronal progenitor cells is isolated.

Another aspect of the present invention relates to a method of producing an enriched or purified population of dopaminergic neuronal progenitor cells from a population of embryonic stem cells. This method involves selecting a promoter or enhancer which functions only in said dopaminergic neuronal progenitor cells and introducing a nucleic acid molecule encoding a marker protein under control of said promoter or enhancer into the population of human embryonic stem cells. The population of embryonic stem cells is induced to produce a mixed population of cells comprising dopaminergic neuronal progenitor cells. The dopaminergic neuronal progenitor cells are allowed to express the marker protein and the cells expressing the marker protein are separated from the mixed population of cells. As a result, an enriched or purified population of dopaminergic neuronal progenitor cells is isolated.

Also encompassed by the present invention is an enriched or purified preparation of isolated dopaminergic neurons derived from embryonic stem cells.

A further aspect of the present invention is an enriched or purified preparation of isolated dopaminergic neuronal progenitor cells derived from embryonic stem cells.

In addition, the present invention is also directed to a cell line of immortalized human mid-brain astrocytes.

The present invention relates to a new strategy for improving the efficiency of dopaminergic neurogenesis from human ES culture, using co-culture with telomerase-immortalized human fetal mesencephalic astrocytes during sonic hedgehog (“SHH”)/FGF8-mediated neuronal induction. By this means, the high efficiency enrichment of dopaminergic neurons is achieved to hitherto unattained purity, with a homogeneity and viability compatible with therapeutic implantation.

The functional utility of these enriched dopaminergic neuronal pools was assessed by transplanting these cells into 6-hydroxydopamine (“6-OHDA”) lesioned adult rat brain. This was done to test the hypothesis that by virtue of their greater homogeneity, these hES-derived dopaminergic neurons would permit improved benefit accruing to intrastriatal transplantation. It was found that when engrafted into the striata of 6-OHDA-lesioned mice, these cells indeed mediated the restitution of motor function in the engrafted animals, relative to their sham-operated controls. However, it was also noted that the grafts exhibited phenotypic instability, with central diminution of dopaminergic neurons and persistent proliferation of undifferentiated engrafted cells.

These results suggest the potential utility of mesencephalic astroglial co-culture in driving dopaminergic neurogenesis from human ES cells, while demonstrating the potential of these cells to mediate neurological improvement in an experimental model of nigrostriatal loss. These findings also suggest the need for caution in this approach, given the phenotypic instability and potential for undifferentiated donor cell expansion that was observed, often in many of the same animals that exhibited neurological improvement after dopaminergic engraftment.

### BRIEF DESCRIPTION OF THE DRAWINGS

FIG. 1 shows a schematic summary of one embodiment of the process of the present invention, outlining the major steps involved in generating and isolating dopaminergic neurons from human ES cells, as well as the preferred sequence for these steps.

FIGS. 2A-D show that human embryonic stem cells may be neuralized by FGF2, providing a cellular substrate for dopaminergic neurogenesis. Human ES cells of the H9 line are neuralized by expansion in bFGF in adherent monolayer culture, in feeder-free conditions using either mouse embryonic fibroblast conditioned media. It is noted that nominally humanized conditions may be achieved using media conditioned by human foreskin fibroblasts. The resultant neuralized cells may then be used as a substrate for specific dopaminergic induction and selection.

FIGS. 3A-G show that dopaminergic neurons can be generated from hES cells. FIG. 3A shows the schematic outline of the stages followed for the induction and differentiation of dopaminergic neurons from hES cells. This procedure begins with undifferentiated human embryonic stem cells which are then used as a substrate for neuralization followed by dopaminergic induction. FIGS. 3B-C show the formation of rosettes at stage 3. These rosettes (FIG. 3B) primarily expressed nestin and were highly mitotic, as indicated by BrdU incorporation, Some spontaneous differentiation to neurons could be seen at the edge of the rosettes (FIG. 3C). FIGS. 3D-F show the induction of DA progenitors at stage 4, as indicated by the presence of engrailedl (Eng1) (FIG. 3D), Pax2, and Otx2. FIG. 3G show that at stage 5, dopaminergic neurons, as indicated by the expression of TH by βIII-tubulin positive neurons were seen.

FIGS. 4A-F show SHH/FGF8 was necessary but not sufficient for high-grade dopaminergic enrichment among newly generated neurons. SHH/FGF8 induction modestly increased the proportion of TH⁺ neurons in human ES cultures, in this case from a baseline of 5.2±3.4% to 15.6±2.9% by 3 weeks after initial SHH/FGF8 exposure. This figure rose to >25% with longer incubation periods, but the concurrent co-culture with hMAST-TERT human mesencephalic astrocytes yielded a >70% incidence of dopaminergic neurons.

FIGS. 5A-B show that retroviral transduction with hTERT can be used to immortalize and select a line of telomerase-overexpressing human fetal mesencephalic astrocytes. A line of human midbrain astrocytes was established to attempt to potentiate the effects of SHH and FGF8 in inducing dopaminergic phenotype by neuralized human ES cells. In order to have a homogenous and stable line for this purpose, a retroviral vector encoding human telomerase reverse transcriptase (“hTERT”) was used to stably immortalize these cells. The resultant line, hMAST-TERT, was generated by using retroviral-hTERT-IRES-puro to stably immortalize human fetal mesencephalic astrocytes derived from an 11 week gestational age fetus. After puromycin antibiotic selection, the line was propagated through serial passage; as shown in FIG. 5A, the line had undergone approximately 56 PDs; as shown in FIG. 5B, its cells homogeneously expressed the astrocytic marker glial fibrillary acidic protein.

FIGS. 6A-D show that co-culture with the hMAST line of telomerase-immortalized human fetal midbrain astrocytes potentiated the inductive effects of FGF8/SHH in human ES cultures and increased the proportion of dopaminergic neurons generated from hES cells. FIG. 6A-D show low and high power images of βIII-tubulin positive neurons expressing tyrosine hydroxylase (“TH”). A high proportion of hES-derived neurons became dopaminergic when co-cultured with a midbrain astrocyte line generated from a 22 week human fetus.

FIGS. 7A-D show hMAST co-culture potentiates dopaminergic neurogenesis. FIGS. 7A-B show graphs of the percentage of βIII-tubulin expressing neurons (FIG. 7A) and the percentage of βIII-tubulin-expressing neurons that express TH (FIG. 7B). Each bar represents a different combination of induction and differentiation conditions in which the cells were cultured in stages IV and V. FIGS. 7C-D show graphs of a comparative effect of midbrain astrocytes and cortical astrocytes on the proportion of βIII-tubulin-expressing neurons and the percentage of TH⁺ among the βIII-tubulin-expressing neurons generated from hES cells.

FIG. 8 shows human fetal astrocytic co-culture potentiated the SHH/FGF8-mediated induction of dopaminergic neurons from human ES cells. This image shows a culture of ES cell-derived dopaminergic neurons, co-expressing tyrosine hydroxylase and βIII-tubulin, derived from H9 human ES cells treated with SHH/FGF8 and raised in the presence of hMAST-TERT cells. Virtually all of the βIII-tubulin-defined neurons in this culture are dopaminergic, as defined by tyrosine hydroxylase expression 3 weeks after SHH/FGF8 induction.

FIGS. 9A-D show the behavioral improvement of 6-OHDA-lesioned rats treated with dopaminergic cell grafts raised in presence of hMAST cells. FIG. 9A is a graph, showing the effect of 6-OHDA concentration on the level of DA in the rat striatum. FIG. 9B shows an analysis of apomorphine-induced rotations with the scores of apomorphine-induced rotations in animals transplanted with hES cells (n=6) and control (saline) (n=5). The transplanted rats showed a decrease in number of rotations with time (p<0.01). FIG. 9C is a graph showing data from the adjusting steps test. A number of adjusting steps was taken during 5 sec for 0.9 m, in the backhand direction, for each forelimb. The transplanted rats performed equally with either paw, while the non-transplanted rats used the contralateral paw more than the ipsilateral one (***p<0.001). FIG. 9D shows data from the cylinder test, in which the number of times each forelimb was used for landing after rearing for 10 minutes was scored. Both groups used either paw with equal frequency.

FIG. 10A-D show hES cell derived progenitors engraft and differentiate as TH-expressing neurons in the striatum of 6-OHDA lesioned rats. FIG. 10A shows dopaminergic differentiation could be seen as early as 4 weeks after transplant. Transplanted cells engrafted extensively in the striatum and on analysis of serial sections were observed to have spread 1.35±0.31 mm across the mediolateral axis. Shown here, in the sagittal plane, is a low power image of a xenograft localized with antibody to human nuclear antigen (“hNA”). A majority of the TH⁺ neurons can be observed at the graft edge. FIGS. 10B-C show high power images of TH-expressing neurons observed at the xenograft edge. FIG. 10D shows that while most of the cells remained close to the transplant core, some TH-expressing neurons could be seen migrating away from the xenograft core.

FIGS. 11A-B show that transplanted cells engraft extensively in the striatum of 6-OHDA lesioned rats. FIG. 11A shows a cartoon of the actual distribution of transplanted cells in sagital rat brain sections at different points along the media-lateral axis. In this rat brain, cell engraftment could be observed spanning a distance of 2.51 mm. FIG. 11B is a graph showing the average density/mm³ of total engrafted cells and engrafted cells that were TH⁺ in 6 rat brains. Of note, the density of tyrosine hydroxylase neurons exceeded 20,000/mm³ within the transplant bed.

FIGS. 12A-E show the dopaminergic differentiation among the engrafted cell population was associated with host astrocyte activity. Pronounced co-localization was observed between regions of host glial response and sustained dopaminergic differentiation. FIGS. 12A-B show low power images of the entire xenograft (hNA). A dense population of reactive host astrocytes (GFAP) can be seen localized in and around the edges of the xenograft. Note the low density and low morphological complexity of the GFAP-expressing host astrocytes in areas away from the xenograft. FIG. 12C is a high power image of host astrocytes interspersed with nuclei (“hNA”) of xenografted cells (boxed area in FIG. 12A). FIG. 12D depicts an adjacent section showing the presence of TH-expressing neurons overlapping with areas rich in reactive host astrocyte (boxed area in FIG. 12A). FIG. 12E shows the percentage of cells expressing TH in different areas of the xenograft. A significantly higher relative proportion of TH⁺ neurons, 65.0±10.0%, was observed within the graft periphery, in proximity to reactive host astrocytes (bar).

FIGS. 13A-E show that xenografted brains had no tumor formation despite persistent undifferentiated expansion. FIG. 13A shows that at ten weeks after transplantation, no tumor formation was observed in any of the grafted animals. Hematoxylin and eosin-stained view of graft border. FIG. 13B-D show an average of 6.6±1.6% of the engrafted cells incorporated BrdU (FIG. 13C), while less than 1% expressed histone-3 (FIG. 13D), a marker for cells undergoing active division. FIG. 13E shows that hES donor-derived nestin-expressing cells were abundant in grafts at 10 weeks.

FIG. 14 shows that the immunostaining of an 11-week fetal human mesencephalic/midbrain revealed clusters of neurogenin2-immunorecative cells in the ventricular zone (“VZ”). Also shown is a cell migrating from the VZ, which expresses the transcription factor engrailed1 (“engr1”) which is expressed by post-mitotic midbrain neurons that include the dopaminergic pool. These include the progenitors of the substantia nigra, which include the dopaminergic neuronal nigrostriatal neurons.

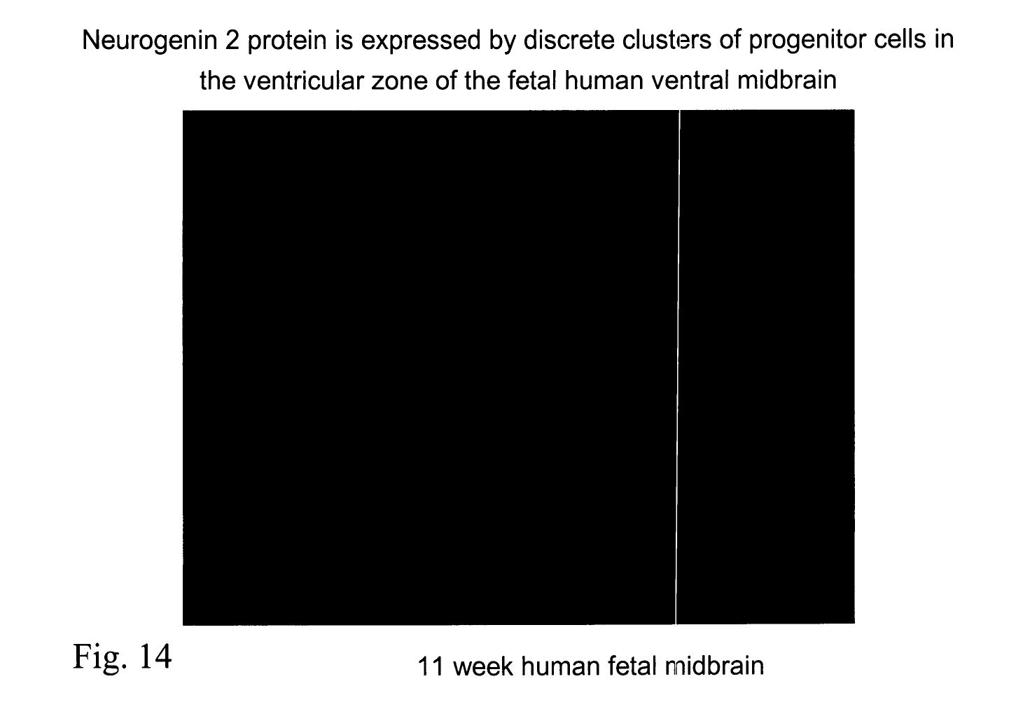

FIGS. 15A-B show a 4.4 kb 5′ enhancer within the neurogenin2 gene directs gene expression to ventral mesencephalic cells. This segment (TgN2-2) of DNA also directs gene expression to neurons of the ventral spinal cord, including motor neurons (Simmons et al., “Neurogenin2 Expression in Ventral and Dorsal Spinal Neural Tube Progenitor Cells is Regulated by Distinct Enhancers,” Dev. Biol. 229:327-39 (2001), which is hereby incorporated by reference).

FIG. 16 shows the use of the 4.4 kb neurogenin2 enhancer to direct GFP expression to dopaminergic progenitors within human midbrain cultures. The E/ngn2 (4.4 kb):β-globin promoter:EGFP used to construct a transgenic E/ngn2:GFP mouse (see FIG. 16). The construct was then used to transfect dissociates of fetal human midbrain (FIG. 17) and SHH/FGF8-induced hES cells (FIG. 18), so as to establish the phenotypic specificity of the enhancer construct, as well as its ability to direct gene expression to ventral midbrain progenitors in human as well as murine cells.

FIGS. 17A-I show that the 4.4 kb neurogenin2 enhancer directed GFP expression to both ventral mesencephalic and spinal neuronal progenitors in transgenic mice. Transgenic mice were established with E/neurogenin2 placed upstream to a β-globin basal promoter driving EGFP. By E10 days, GFP expression was noted in both the ventral midbrain and throughout much of the ventral spinal cord.

FIGS. 18A-H show fetal human midbrain progenitor cells expressed E/ngn2-driven GFP. Human fetal mesencephalic dissociates prepared from first trimester human midbrain of E11.5 week gestational age, transfected with E/ngn2:P/β-globin:EGFP. Over a 1-3 day time course, GFP fluorescence revealed the presence of many E/ngn2:EGFP⁺ cells. These cells went on to express tyrosine hydroxylase, consistent with nascent dopaminergic phenotype.

FIGS. 19A-B show that SHH/FGF8-/hMAST co-cultured human ES cells expressing GFP under the control of the 4.4 kb neurogenin 2 enhancer were largely dopaminergic progenitor cells. When transfected into SHH/FGF8-treated human ES cells transfected by electroporation with E/ngn2:EGFP plasmid DNA and raised in noncontiguous co-culture with hMAST-TERT human midbrain astrocytes, a subpopulation of neurons developed neurogenin2-regulated GFP expression. These cells went on to express tyrosine hydroxylase as a marker of dopaminergic phenotype.

FIGS. 20A-B show the flow cytometry for GFP⁺ cells among E/ngn2:EGFP-transfected hES cells. After correction for transfection efficiency, 26.6% of E/Ngn2⁺ cells in SHH/FGF8-treated and HMAST-TERT exposed hES cultures expressed GFP from the ngn2 enhancer. (Ngn2:GFP⁺ incidence (8.5%)/transfection efficacy (0.32)=26.6%). This corresponded to the net incidence of TH⁺ cells in these cultures at this timepoint.

### DETAILED DESCRIPTION OF THE INVENTION

The present invention relates to a method of isolating an enriched or purified population of dopaminergic neuronal progenitor cells from a population of embryonic stem cells. This involves providing a population of embryonic stem cells and inducing production of dopaminergic neuronal progenitor cells from the population of embryonic stem cells. A promoter or enhancer which functions only in dopaminergic neuronal progenitor cells is selected and a nucleic acid molecule encoding a marker protein under control of said promoter or enhancer is introduced into the induced population of embryonic stem cells. The dopaminergic neuronal progenitor cells are allowed to express the marker protein, and the cells expressing the marker protein are separated from the induced population of embryonic stem cells. As a result, an enriched or purified population of dopaminergic neuronal progenitor cells is isolated.

As used herein, the term “isolated” when used in conjunction with a nucleic acid molecule refers to: 1) a nucleic acid molecule which has been separated from an organism in a substantially purified form (i.e. substantially free of other substances originating from that organism), or 2) a nucleic acid molecule having the same nucleotide sequence but not necessarily separated from the organism (i.e. synthesized or recombinantly produced nucleic acid molecules).

The term “enriched” refers to a cell population that is at least 90% pure with respect to the index phenotype, regardless of its initial incidence in the population from which it was derived. The term “purified” refers to a cell population at least 99% pure with respect to the index phenotype, regardless of its initial incidence in the reference population.

Preferably, these embryonic stem cells are of human origin. In carrying out the method of the present invention, those cells are kept in cell culture. In a preferred embodiment, the culture also contains astrocytes and, it is particularly preferred if the astrocytes are human mid-brain astrocytes, most preferably immortalized, human mid-brain astrocytes. Immortalization can be carried out by transduction with a nucleic acid molecule encoding a human telomerase reserve transcriptase (“hTERT”). Such nucleic acid molecules are described in U.S. Pat. No. 6,166,178 to Cech et al., which is hereby incorporated by reference in its entirety. Transduction with a nucleic acid molecule encoding a human telomerase reverse transcriptase is achieved in substantially the same manner as is used to transduce cells with a marker protein-encoding nucleic acid molecule, except that any promoter (not necessarily a cell-specific promoter) which will achieve expression can be used. As a result, telomerase-immortalized lines of astrocytes can be established. These astrocytes can be fetal-derived or adult-derived.

The present invention can be carried out using a promoter or enhancer that functions in and a nucleic acid encoding a marker protein, as described in U.S. Pat. No. 6,245,564 to Goldman et. al., which is hereby incorporated by reference in its entirety. In particular, this involves providing embryonic stem cells and selecting a promoter or enhancer which functions only in dopaminergic neuronal progenitor cells and not in other cell types. A nucleic acid molecule encoding a marker protein under control of the promoter or enhancer is introduced into the embryonic stem cells but not the other cell types, are allowed to express the marker protein. The cells expressing the marker protein are identified as being restricted to dopaminergic neuronal progenitor cells and are separated from the mixed population to produce an isolated population of dopaminergic neuronal progenitor cells.

Any promoter or enhancer which is specific for dopaminergic neurons or their progenitors can be utilized in this process. “Specific”, as used herein to describe a promoter or enhancer, means that the promoter or enhancer functions only in the chosen cell type. Suitable promoters or enhancers include neurogenin-2 (Simmons et al., “Neurogenin2 Expression in Ventral and Dorsal Spinal Neural Tube Progenitor Cells is Regulated by Distinct Enhancers,” Dev. Biol 229(2):327-29 (2001) and Zhou et al., “The bHLH Transcription Factors OLIG2 and OLIG1 Couple Neuronal and Glial Subtype Specification,” Cell 109(1):67-73 (2002), which are hereby incorporated by reference in their entirety); promoters/enhancers for genes in the dopamine synthesis pathway, including tyrosine hydroxylase, dopadecarboxylase, aromatic decarboxylase, and dopamine β-hydroxylase; promoters/enhancers for dopamine transport proteins such as the dopamine transporter (“DAT”); and promoters/enhancers for genes expressed differentially in the ventral mid-brain, such as engrailed-1 and nurr-1. See Castel-Branco et al., “Differential Regulation of Midbrain Dopaminergic Neuron Development by Wnt-1, Wnt-3a, and Wnt-5a,” Proc. Natl. Acad. Sci. USA 100(22):12747-52 (2003), which is hereby incorporated by reference in its entirety.

The marker protein is preferably a green fluorescent protein (“GFP”). The isolated nucleic acid molecule encoding a green fluorescent protein can be deoxyribonucleic acid (“DNA”) or ribonucleic acid (“RNA”, including messenger RNA or “mRNA”), genomic or recombinant, biologically isolated or synthetic. The DNA molecule can be a cDNA molecule, which is a DNA copy of a messenger RNA (mRNA) encoding the GFP. In one embodiment, the GFP can be from Aequorea victoria (U.S. Pat. No. 5,491,084 to Chalfie et al., which are hereby incorporated in their entirety). A plasmid designated pGFP10.1 has been deposited pursuant to, and in satisfaction of, the requirements of the Budapest Treaty on the International Recognition of the Deposit of Microorganisms for the Purposes of Patent Procedure, with the American Type Culture Collection (ATCC), 12301 Parklawn Drive, Rockville, Md. 20852 under ATCC Accession No. 75547 on Sep. 1, 1993. This plasmid is commercially available from the ATCC and comprises a cDNA which encodes a green fluorescent protein of Aequorea Victoria as disclosed in U.S. Pat. No. 5,491,084 to Chalfie et al., which is hereby incorporated in its entirety. A mutated form of this GFP (a red-shifted mutant form) designated pRSGFP-C1 is commercially available from Clontech Laboratories, Inc. (Palo Alto, Calif.).

Mutated forms of GFP that emit more strongly than the native protein, as well as forms of GFP amenable to stable translation in higher vertebrates, are now available and can be used for the same purpose. Alternatively, the GFP can be in humanized form (“GFPh”) (Levy, J., et al., Nature Biotechnol. 14:610-614 (1996), which is hereby incorporated in its entirety). Any nucleic acid molecule encoding a fluorescent form of GFP can be used in accordance with the subject invention.

Other suitable marker proteins include lacZ/beta-galactosidase or alkaline phosphatase.

Standard techniques are then used to place the nucleic acid molecule encoding marker protein-encoding nucleic acid molecule under the control of the chosen cell specific promoter. Generally, this involves the use of restriction enzymes and ligation.

The resulting construct, which comprises the nucleic acid molecule encoding the marker protein under the control of the selected promoter or enhancer (themselves a nucleic acid molecule) (with other suitable regulatory elements if desired), is then introduced into a plurality of cells which are to be sorted. Techniques for introducing the nucleic acid molecules of the construct into the plurality of cells may involve the use of expression vectors which comprise the nucleic acid molecules. These expression vectors (such as plasmids and viruses) can then be used to introduce the nucleic acid molecules into the plurality of cells.

Various methods are known in the art for introducing nucleic acid molecules into host cells. These include: 1) microinjection, in which DNA is injected directly into the nucleus of cells through fine glass needles; 2) dextran incubation, in which DNA is incubated with an inert carbohydrate polymer (dextran) to which a positively charged chemical group (DEAE, for diethylaminoethyl) has been coupled. The DNA sticks to the DEAE-dextran via its negatively charged phosphate groups. These large DNA-containing particles stick in turn to the surfaces of cells, which are thought to take them in by a process known as endocytosis. Some of the DNA evades destruction in the cytoplasm of the cell and escapes to the nucleus, where it can be transcribed into RNA like any other gene in the cell; 3) calcium phosphate coprecipitation, in which cells efficiently take in DNA in the form of a precipitate with calcium phosphate; 4) electroporation, in which cells are placed in a solution containing DNA and subjected to a brief electrical pulse that causes holes to open transiently in their membranes. DNA enters through the holes directly into the cytoplasm, bypassing the endocytotic vesicles through which they pass in the DEAE-dextran and calcium phosphate procedures (passage through these vesicles may sometimes destroy or damage DNA); 5) liposomal mediated transformation, in which DNA is incorporated into artificial lipid vesicles, liposomes, which fuse with the cell membrane, delivering their contents directly into the cytoplasm; 6) biolistic transformation, in which DNA is absorbed to the surface of gold particles and fired into cells under high pressure using a ballistic device; and 7) viral-mediated transformation, in which nucleic acid molecules are introduced into cells using viral vectors. Since viral growth depends on the ability to get the viral genome into cells, viruses have devised efficient methods for doing so. These viruses include retroviruses, lentivirus, adenovirus, herpesvirus, and adeno-associated virus.

As indicated, some of these methods of transforming a cell require the use of an intermediate plasmid vector. U.S. Pat. No. 4,237,224 to Cohen and Boyer, which is hereby incorporated in its entirety, describes the production of expression systems in the form of recombinant plasmids using restriction enzyme cleavage and ligation with DNA ligase. These recombinant plasmids are then introduced by means of transformation and replicated in unicellular cultures including procaryotic organisms and eucaryotic cells grown in tissue culture. The DNA sequences are cloned into the plasmid vector using standard cloning procedures known in the art, as described by Sambrook et al., Molecular Cloning: A Laboratory Manual, 2d Edition, Cold Spring Harbor Laboratory Press, Cold Spring Harbor, N.Y. (1989), which is hereby incorporated in its entirety.

In accordance with one of the above-described methods, the nucleic acid molecule encoding the GFP is thus introduced into a plurality of cells. The promoter or enhancer which controls expression of the GFP, however, only functions in the cell of interest. Therefore, the GFP is only expressed in the cell of interest. Since GFP is a fluorescent protein, the cells of interest can, therefore, be identified from among the plurality of cells by the fluorescence of the GFP.

The inducing step typically involves administering an inducer like FGF2, FGF8, FGF20, sonic hedgehog (“SSH”), and mixtures thereof. Such administration can be carried out by direct administration in vitro.

Any suitable means of detecting the fluorescent cells can be used. The cells may be identified using epifluorescence optics, and can be physically picked up and brought together by Laser Tweezers (Cell Robotics Inc., Albuquerque, N. Mex.) They can be separated in bulk through fluorescence activated cell sorting, a method that effectively separates the fluorescent cells from the non-fluorescent cells.

Once the enriched or purified population of dopaminergic neuronal progenitor cells is isolated in accordance with the present invention, those neuronal progenitor cells can be transplanted into a subject. This may be accomplished either by transuterine intra-embryonic or intra-fetal injection, either into the spinal cord, brain stem, or brain; or by post-natal injection directly into these same areas. Such transplantation is expected to be beneficial in treating subjects with dopamine-depletion disease, such as Parkinson's Disease.

Alternatively, the enriched or purified population of dopaminergic neuronal progenitor cells can be differentiated into dopamineric neurons. This is achieved by culturing with growth factor supplementation with one or more of the following: serum, brain-derived neurotrophic factor, glial-derived neurotrophic factor, and neurotrophic factor-4.

Another aspect of the present invention relates to a method of producing an enriched or purified population of dopaminergic neuronal progenitor cells from a population of embryonic stem cells. This method involves selecting a promoter or enhancer which functions only in said dopaminergic neuronal progenitor cells and introducing a nucleic acid molecule encoding a marker protein under control of said promoter or enhancer into the population of human embryonic stem cells. The population of embryonic stem cells is induced to produce a mixed population of cells comprising dopaminergic neuronal progenitor cells. The dopaminergic neuronal progenitor cells are allowed to express the marker protein and the cells expressing the marker protein are separated from the mixed population of cells. As a result, an enriched or purified population of dopaminergic neuronal progenitor cells is isolated.

In carrying out this aspect of the present invention, substantially the same reagents and steps described above are utilized.

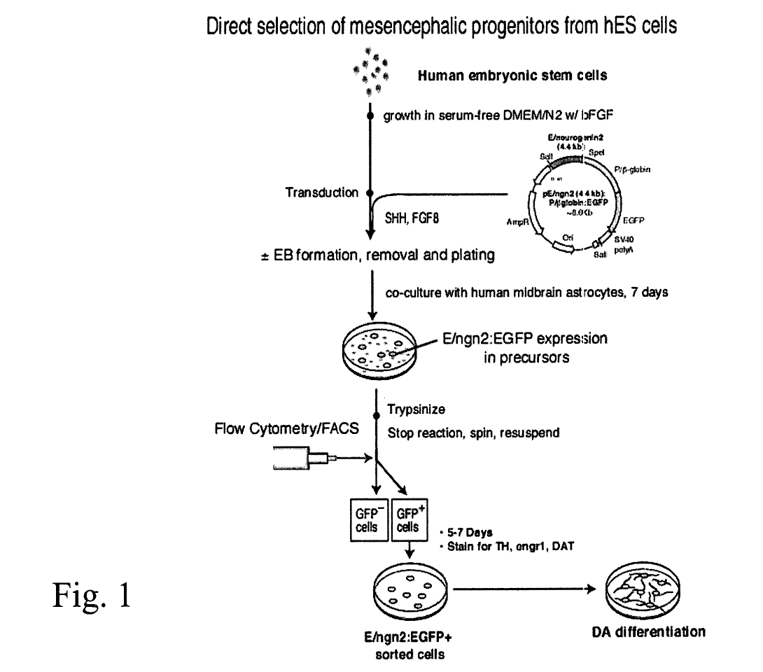

This process is illustrated in FIG. 1 which shows the direct selection mesencephalic progenitors from hES cells. As shown in that drawing, human embryonic stem cells are grown in serum-free DMEM/N2 with bFGF. These cells are then transduced with the pE/ngn2:P/βglobin:EGFP vector in the presence of sonic hedgehog and FGF8. After the cells are removed and plated, they are co-cultured with human mid-brain astrocytes. Cells expressing E/ngn2:EGFP are recovered by fluorescent activated cell sorting/flow cytommetry. The E/ngn2:EGFP⁺ cells can then undergo differentiation.

Also encompassed by the present invention is an enriched or purified preparation of isolated dopaminergic neurons derived from embryonic stem cells and an enriched or purified preparation of isolated dopaminergic neuronal progenitor cells derived from embryonic stem cells. This preparation is formulated, respectively, from the dopaminergic neurons and dopaminergic neuronal progenitor cells described above. These cells are characterized by their high degree of enrichment of dopaminergic neurons or progenitors thereof, including the capability of being purified with the concurrent abolition of any undifferentiated or inappropriately differentiated cells from the final product. Furthermore these cells are generated from embryonic stem cells without contacting non-human cells or agents, allowing them to be free of non-human agents—i.e. the cells are humanized. As a result, these cells are an appropriate vehicle for human transplantation. Further, the preparation of these cells is of human origin and is at least 90% pure, preferably at least 99% pure.

### EXAMPLES

### Example 1

### hES Culture

H9 hES cells (obtained from Geron Corp; CA) were cultured on matrigel coated (Becton and Dickinson, USA) plates. The cells were fed every day with FGF-2 (4 ng/ml; Invitrogen, USA) supplement conditioned medium obtained from irradiated mouse embryonic feeder cells (MEFs). The hES cells were passed every 7-14 days or when they were 80% confluent. At each pass, the cells were treated with collagenase type IV (200 U/ml; Invitrogen, USA) for 10 min, and gently scraped from the culture dish. The cells were then split 1:3-1:4, onto Matrigel-coated 6 well plates. The cells were fed every 24 hr with KO-DMEM (Invitrogen) supplemented with 20% KO-Serum replacement (KO-medium), and bFGF (4 ng/ml; Invitrogen). MEF cells were prepared as follows: fibroblasts obtained from E14 mouse embryos were grown to confluency in gelatin (0.1% W/V) coated flasks in DMEM supplemented with 10% fetal bovine serum (FBS; HyClone, USA). The cells were collected by treatment with trypsin/EDTA (Invitrogen) and irradiated at 4000 rads. 4 million irradiated cells were then plated per gelatin-coated T75 flask (Corning), in DMEM supplemented with 10% FBS. 24 hrs later, the irradiated MEFs were fed with 25 ml of KO-medium supplemented with FGF2 (4 ng/ml).

### Example 2

### Induction and Differentiation of DA Neurons

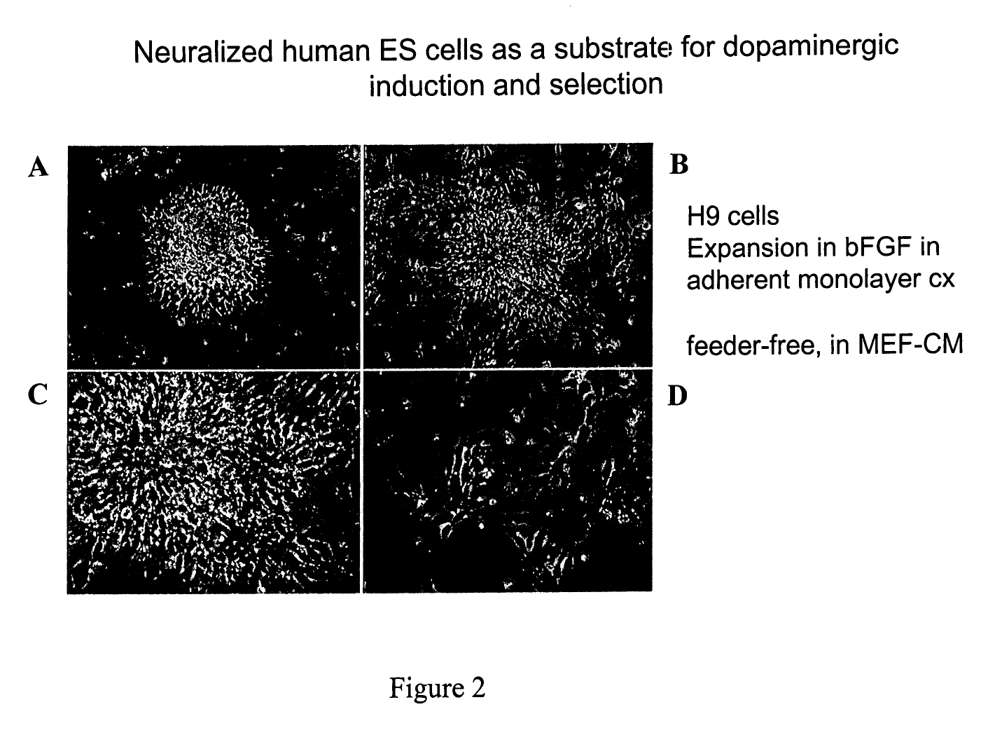

Dopaminergic (“DA”) neurons were induced from hES cells using a protocol modified from McKay, Studer, and colleagues (Lee et al., “Efficient Generation of Midbrain and Hindbrain Neurons From Mouse Embryonic Stem Cells,” Nature Biotechnol 18-675-679 (2000); Pernier et al. “Derivation of Midbrain Dopamine Neurons From Human Embryonic Stem Cells,” Proc Natl Acad Sci USA 101:12543-12548 (2004), which are hereby incorporated by reference in their entirety) (FIG. 2A). Briefly, at 80% confluency, hES cell cultures were dissociated to form embryoid bodies (“EBs”). EBs were generated by scraping collagenase-treated hES cells and suspending them in Ultralow cluster 6 well plates (Corning, USA) in KO-medium without FGF2 for 4 days. EBs thus obtained were mechanically triturated and plated on tissue culture dishes in serum-free media supplemented with insulin/transferrin/selenite and fibronectin (ITSF medium), as described in Okabe et al., “Development of Neuronal Precursor Cells and Functional Postmitotic Neurons From Embryonic Stem Cells In Vitro,” Mech Dev 59:89-102 (1996), which is hereby incorporated by reference in its entirety. The cultures were maintained in this medium for 10 days, with fresh medium changes every 3 days.

For induction of DA progenitors, cells were collected by a combination of trypsine (0.05%)/EDTA (Invitrogen, USA) treatment and mechanical trituration. The cells were transferred to poly-omithin/laminin (1 μg/ml; BD, USA) coated tissue culture dishes in DMEM/F12 medium supplemented with N2 (1X, Invitrogen, USA), laminin (1 μg/ml), and FGF2 (10 ng/ml). Some cultures were also exposed to the N-terminal active fragment of human sonic hedgehog (“SHH”) (200 ng/ml; generously provided by Curis, Inc., MA) and FGF8 (100 ng/ml; R&D, USA) and cultured in the presence or absence of immortalized hTERT-overexpressing human brain astrocytes (description for astrocyte cultures provided in the subsequent section). Medium with fresh supplements was added ever 3 days.

Dopaminergic neuronal differentiation was induced after 6-8 days by withdrawing SHH and the FGFs, and replacing them with base media supplemented instead with glial-derived neurotrophic factor (“GDNF”) (20 ng/ml), brain-derived neurotrophic factor (“BDNF”) (20 ng/ml), and 0.5% FBS (Basal Medium, BM). In some wells, fresh inserts of hMAST-TERT midbrain astrocytes were also added. For immunocytochemical assessment of dopaminergic differentiation, cultures were fixed 7-14 days later. For transplantation, cultures were used after 2 days in differentiation conditions.

### Example 3

### Generation of Immortalized hTERT-Overexpressing Astrocyte Lines

Midbrain or cortical tissues were dissected out from human 22 week gestational brains in Ca²⁺/Mg²⁺-free Hanks Balanced Salt Solution (“HBSS”). The tissue was dissociated as per established protocols (Roy et al., “Telomerase Immortalization of Neuronally Restricted Progenitor Cells Derived from the Human Fetal Spinal Cord,” Nature Biotechnology 22:297-30 (2004), which is hereby incorporated by reference in its entirety). In short, dissected tissue was minced, suspended in PIPES buffer (in mM: 120 NaCl, 5 KCl, 25 glucose, and 20 PIPES), then digested in papain-PIPES (11.4 U/ml papain; Worthington, Freehold, N.J.) and DNase I (10 U/ml; Sigma, St. Louis, Mo.) for 40 min at 37° C. The tissue was collected by centrifugation and resuspended in DMEM/F-12 supplemented with N1 (Sigma, USA) in the presence of DNase I and reincubated for 15-30 min at 37° C. The tissue was finally dissociated by sequentially triturating 20, 10, and 5 times through glass Pasteur pipettes fire-polished to decreasing bore diameters. The cells were passed through a 40 μm mesh into DMEM/F-12/N1 medium with 10% fetal bovine serum (FBS, Hyclone, USA) to stop the enzymatic dissociation. The cells were then suspended in DMEM/F12/N1, supplemented with 10% FBS and plated in 6 well tissue culture dishes at a density of 1 million cells per well.

For immortalization by hTERT-overexpression, cells were infected with VSVg-pseudotyped retrovirus encoding hTERT in pBABE-puro under CMV control (Roy et al., “Telomerase Immortalization of Neuronally Restricted Progenitor Cells Derived from the Human Fetal Spinal Cord,” Nature Biotechnology 22:297-30 (2004, which is hereby incorporated by reference in its entirety). The cells were treated with fresh viral supernatant every 8-hours for 1 day in the presence of 8 μg/ml polybrene. Following viral infection, the cells were plated at lower densities to decrease the number of transfectants per culture dish. 1-2 weeks after the viral infection, positively transduced cells were selected in 1 μg/ml puromycin (Sigma) for 2 weeks. Successful selectants were isolated using sterile cloning rings and propagated in DMEM/F12/N1/10% FBS. Once lines were propagated in sufficient numbers, their astrocytic identity was confirmed by immunocytochemistry for GFAP, A2B5, and S100beta. Several aliquots were also frozen for future use.

For use in co-cultures with hES cells, the immortalized astrocytes were cultured on 0.22 micron co-culture inserts (Corning, USA) for 3 days in DMEM/F12/N1/10%FBS or till they were 70-80% confluent. Before co-culturing with hES cells, the inserts with astrocytes were rinsed with DMEM/F12. After every 7 days in co-culture, the old inserts were replaced with fresh astrocyte containing inserts.

### Example 4

### 6-hydroxydopamine Lesioning

Adult male Sprague Dawley rats (8 weeks old, 250 g, Taconic) were used for transplantation studies. Animals were housed and treated according to the NIH guidelines. They were maintained in a temperature/humidity-controlled environment under a 12 hr light/dark cycle with free access to food and water.

To determine the optimum dose of 6-OHDA (Sigma) needed to elicit maximum striatal DA loss without affecting mortality, the 6-OHDA dose was titrated against striatal DA level and mortality, in a pilot series. In this set of experiments, rats were anesthetized with ketamine/xylazine (100 mg/kg and 10 mg/kg, respectively, i.p.) before being injected with 6-OHDA-HBr into the right ventricle (AP: −1; ML: −1.5; DV: −3.2). The 6-OHDA-HBr was dissolved in 0.09% of NaCl containing 0.05% ascorbic acid at concentrations of 50, 100, or 200 μg/7 μl into the right-ventricle. One month after lesion, the rats were sacrificed by decapitation.

### Example 5

### Determination of DA Level by HPLC

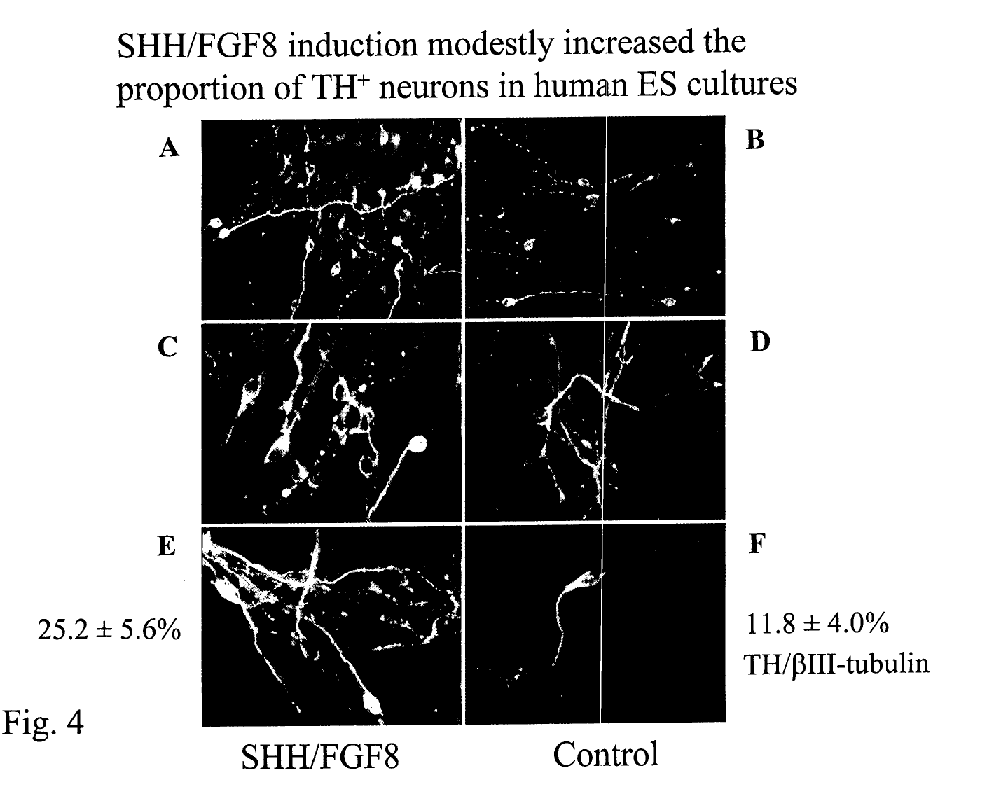

The striata were dissected out and immediately frozen for measurement of DA levels by HPLC. The frozen striata were homogenized by sonication in 0.1 M ice-cold perchloric acid. A 20 microliter aliquot was taken from each sample for protein determination using the Bio-Rad method and Perkin Elmer Bio Assay Reader (Norwalk, Conn.). The samples were centrifuged (14,000 g, 10 min), and the supernatants were centrifuged again (14,000 g, 10 min) and loaded into the HPLC system. The HPLC consisted of a pump and automated injector (515 HPLC and 717 Plus Autosampler, Waters, USA), an analytical colunm (HR80, 4.6 mm×8 cm, 3 μm; ESA, USA), and an electrochemical detector (Coulochem Ill., ESA, USA). Data were recorded and analvzed by a software (ESA 501. USA). For the determination of dopamine, the mobile phase consisted of 75 mM of NaH₂PO₄.H₂O, 1.5 mM OSA, 10% acetonitrile, pH=3. The flow rate was 1 ml/min. Standard solutions containing 500 nM of DA, were injected (20 μl) every 12 samples. Data were recorded and analyzed by ESA 501 (USA). Once the optimum dose of 6-OHDA needed for maximum DA depletion in the striatum was determined (100 μg/7 μl; FIG. 4A), transplantation studies were initiated.

### Example 6

### Cell Transplantation and Immunosuppression

Animals were lesioned with one injection of 100 μg /7 μl of 6-OHDA-HBr into the right ventricle as described in the previous paragraph. Four weeks later, each rat was monitored for 20 minutes to measure apomorphine (2.5 mg/kg, i.p.)-induced rotations. This test measures motor asymmetry. From the results obtained, rats were divided into two equally distributed groups. One week later, rats from one group were transplanted with cells (approximately 500,000 cells/3 μls of HBSS) and another group with saline in striatum ipsilateral to the lesion (AP:0 according to Bregma, ML: −2.8) at 3 different heights (DV: −6, −5, −4). Two days prior to, and 3 days after implantation, rats were immunosuppressed with daily intraperitoneal (IP) injections of Cyclosporine A (20 mg/kg; Sandimmune; Novartis). For the rest of their survival period, the animals received daily Cylosporine A injection of 15 mg/kg.

### Example 7

### Behavioral Testing

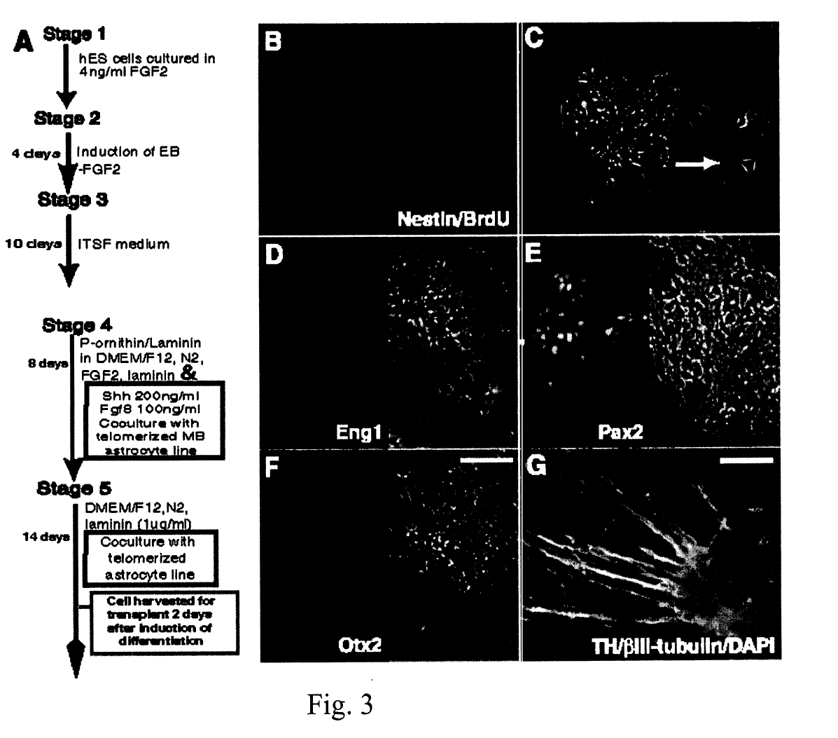

The apomorphine-induced rotation test was repeated four, six, and eight weeks after lesion, in order to check the influence of the graft on motor asymmetry. Nine weeks after grafting, rats were evaluated by the adjusting step test. This test monitors lesion-/transplant-induced changes in forelimb akinesia. The rat was held by the experimenter with one hand fixing the hindlimbs and slightly raising the hind part off the surface, while the other hand fixed the forelimb not to be monitored. The rat was moved sideways (5 sec for 0.9 m) by the experimenter, in the backhand direction, with its free paw touching the table. The number of adjusting steps for each forelimb was measured 3 times per session with 2 sessions a day, for 3 days. During the ninth week; the rats' nerformance was also measured in the cylinder test. This test assesses limb use asymmetry. It measures the level of preference for using the non-impaired forelimb for weight shifting movements during spontaneous vertical exploration. The rat was put in a transparent cylinder (30 cm height, 20 cm diameter) for 10 minutes. The use of each limb was measured when rearing after landing (FIG. 3).

### Example 8

### Immunochemistry

For immunocytochemistry, cultures were fixed with 4% paraformaldehyde for 5 minutes. Animals were perfusion fixed with 4% paraformaldehyde. Their brains were collected and post-fixed for 4 hours in the cold, then cryoprotected in 30% sucrose. Their brains were sectioned at 15 μm by cryostat. Both cells and tissue were permeabilized with 0.1% saponin/1% normal goat serum (“NGS”) for 15 min at RT, and blocked with 0.05% saponin/5% NGS for 15 min. Incubation with primary antibody was for 72 hr at 4° C. Incubation with fluorescently-tagged secondary antibodies was for 2 hr at RT.

The primary antibodies used are as follows: Rabbit anti-human nestin (chemicon) 1:600; muse anti-SSEA4 (ascites; DSHB) 1:100; mouse anti-oct3/4 (ascites; DSHB) 1:100; mouse anti-bill tubulin (Mab TuJ 1, Covance), 1:600; mouse anti-MAP-2 (Mab AP-20, Sigma), 1:100; mouse anti-TH (ImmunoStar, USA), 1:50; rabbit anti-TH (Sigma) 1:1000; mouse anti-Eng1 (ascites, DSHB), 1:100; mouse anti-Pax2 (ascites, DSHB), 1:50; rabbit anti Otx2, 1:1000; rat anti-BrdU antibody (Harlan), 1:200; mouse ant human nuclear antigen (hNA) (1:400, Chemicon). Cells and tissue were counterstained with 4′,6-diamidino-2-phenylindole (DAPI).

### Example 9

### Statistical Analysis

Each set of culture experiment was performed in triplicate wells and each experimental set was repeated a minimum of 3 times. For phenotype quantification, random fields were selected and counting till 500 cells/well was scored per well. Quantification of TH-expressing cells in transplanted rat brains were done by counting the total number of hNA+ nuclei in every 5th sagittal along the media-lateral axis of the right hemisphere. The area of transplanted region was simultaneously determined for each of the scored sections. The percentage of transplanted cells incorporating BrdU or expressing histone-3 was determined by counting 5 random sections at different points along the media-lateral axis. Statistical significance was determined by a one-way ANOVA with Bonferroni t-test. All data is expressed as mean±SEM.

### Example 10

### Dopaminergic Neurons Can Be Generated From hES Cells by Exposure to Shh and FGF8

Human ES cells of the ES line were obtained from Geron and cultured as anchored ES cells, before FGF2-dependent neutralization to stage 2, under feeder-free culture conditions (FIG. 2). Tyrosine hydroxylase-expressing (TH⁺) neurons were generated from hES cultures (H9 line; Geron), as described (FIG. 3A). Phenotypic analysis of cultures after the EB-induction stage showed a gradual change in the population from neural stem cells to differentiated TH-expressing neurons. At stage 3, in the presence of ITSF medium, the formation of small rosettes comprising actively dividing nestin positive cells could be seen (FIG. 3B). Some spontaneous differentiation to neurons could also be seen at the edges of the rosettes (FIG. 3C). When cultures were treated with FGF2/FGF8/SHH in the subsequent stage, cells expressing transcription factors specific to midbrain DA progenitors, which included Pax2, Otx2, and engrailedl (Eng1) (FIGS. 3D-F), were observed. In the final stage, withdrawal of FGF2/FGF8/SHH and inclusion of BDNF/GDNF/0.5%FBS (BM) resulted in differentiation of the DA progenitors to TH⁺ DA neurons (FIG. 3G). In cultures exposed to FGF2/FGF8/SHH in stage 4, 25.2±5.6% (n=3 experiments, each scored in triplicate) of βIII-tubulin⁺ neurons expressed TH, compared to 11.8±4.0% in control cultures supplemented with FGF2 alone (FIG. 4).

### Example 11

### hTERT Transduction Yielded a Stable Line of Human Mesencephalic Astrocytes

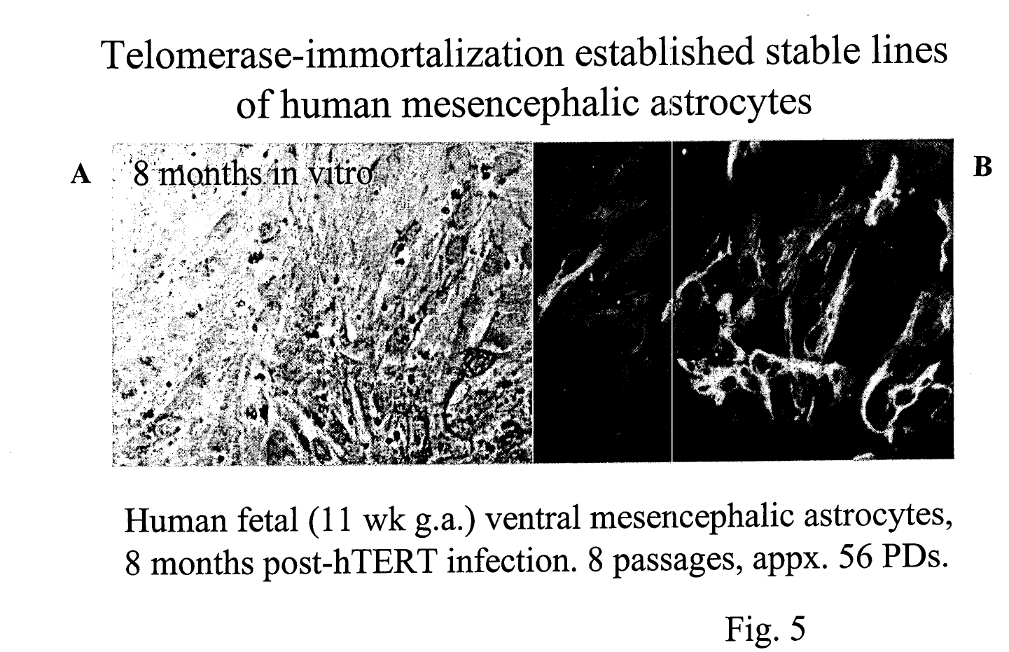

To establish lines of uniform human mesencephalic astrocytes to potentiate the effects of SHH and FGF8 in inducing dopaminergic phenotype by neuralized human ES cells, human fetal midbrain tissue were sampled at 21 weeks gestational age, a period of dopaminergic neuronal maturation and consolidation. The ventral midbrain was dissected and dissociated, then infected with a retrovirus encoding hTERT, under control of the constitutive CMV promoter, up stream to a puro selection cassette under IRES control in pBABE. Stable lines were established after puromycin selection, and characterized antigenically after 3 passages 1 month apart, spanning approximately 21-24 population doublings (PDs) (FIG. 5A). These cells expressed astrocytic GFAP (FIG. 5B) and did not express either early oligodendrocytic (A2B5, 04) or neuronal (βIII-tubulin) markers. They were passaged to 35 PDs before use; these cells have now been maintained for over 2 years in continuous culture as a stable line, which has been designated human midbrain astrocyte-hTERT (hMAST-TERT) cells.

### Example 12

### Telomerase-Immortalized Midbrain Glia Potentiated Dopaminergic Differentiation

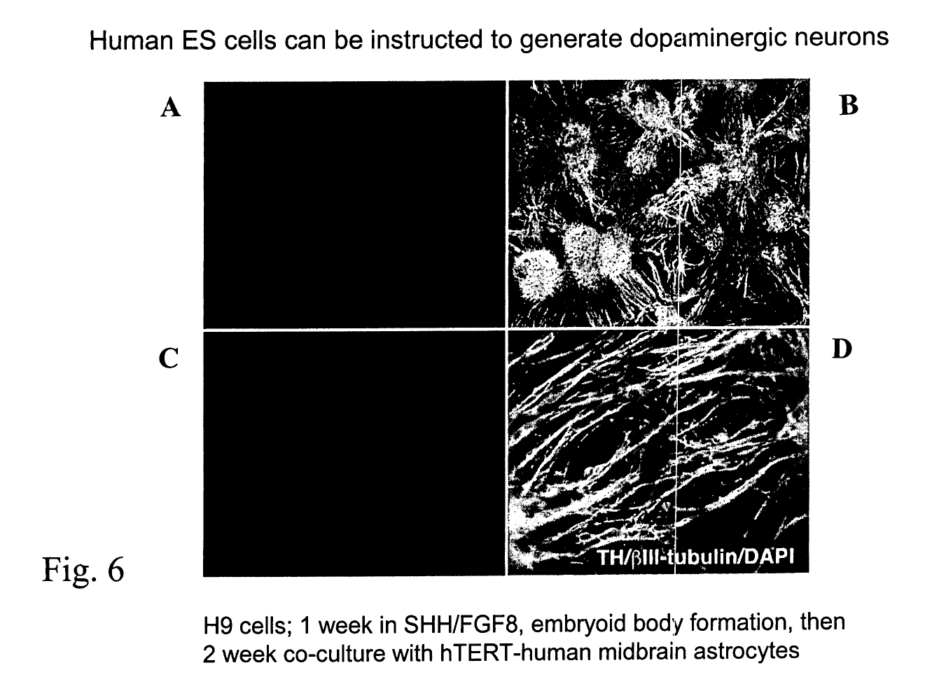

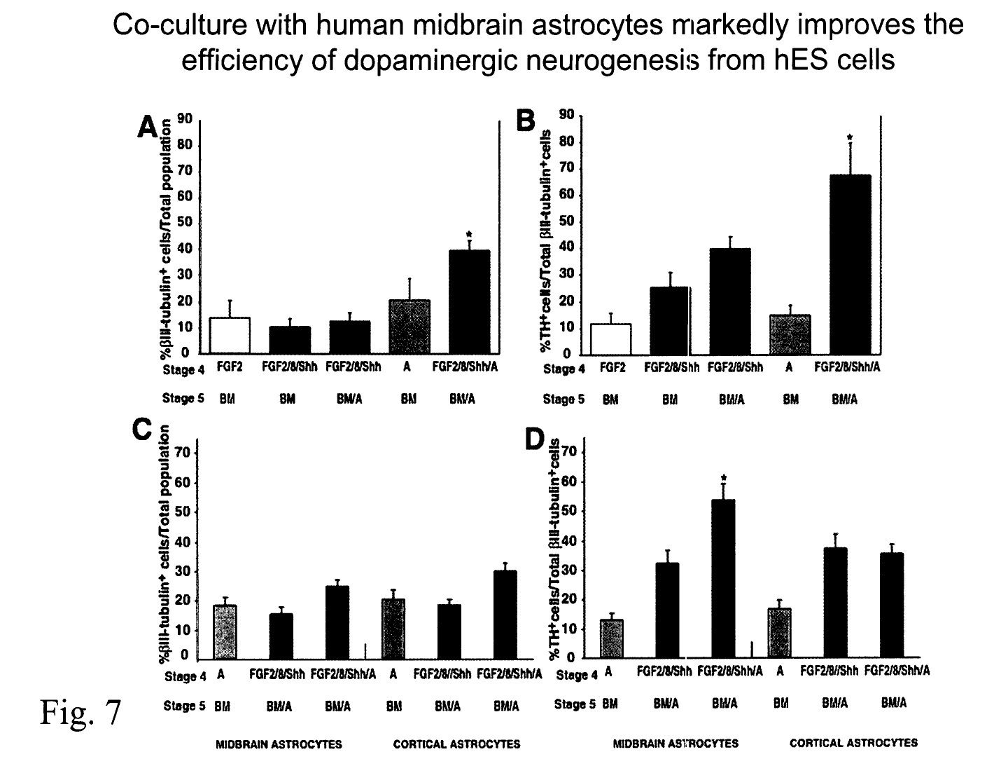

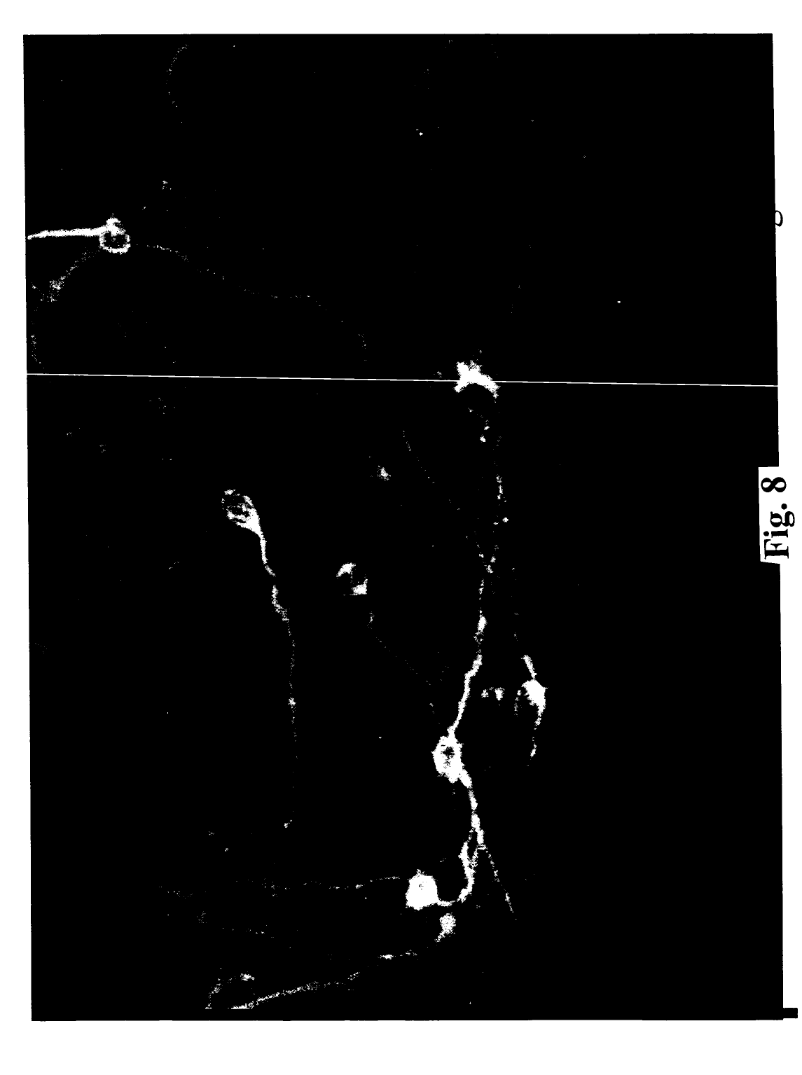

hMAST-TERT cells substantially enhanced tyrosine hydroxylase-defined dopaminergic differentiation from human ES cells (FIGS. 6A-D). When cultures were exposed to FGF2/FGF8/SHH in stage 4, then differentiated in co-cultures with inserts bearing confluent hMAST-TERT midbrain astrocytes, the proportion of TH⁺ neurons increased from 25.2±5.6% to 39.6±4.6% (p<0.05, Student's t-test) (FIGS. 7A-D). More remarkably, when hES derived cells were co-cultured with hMAST-TERT cells from induction throughout differentiation continuously, the proportion of TH⁺ neurons increased to 67.4±12.1% (FIGS. 7B and 8). Significantly, under these conditions, the percentage of βIII-tubulin expressing neurons in the total population also increased to 39.5±3.9%, a significantly higher proportion of neurons than that noted in control cultures maintained in FGF2 or FGF8/FGF2/SHH during stage 4. In addition, hES cells exposed only to hMAST-TERT cells, without benefit of FGF8/FGF2/SHH at stage 4, did not exhibit an increased incidence of TH⁺ neurons relative to cultures maintained only in FGF2 at stage 4, despite manifesting a higher percentage of βIII-tubulin+neurons overall. Together, these data indicate that besides supporting neuronal survival (Takeshima et al., “Mesencephalic Type 1 Astrocytes Rescue Dopaminergic Neurons from Death Induced by Serum Deprivation,” J Neurosci 14:4769-4779 (1994), which is hereby incorporated by reference in its entirety), mesencephalic astrocytes also contributed to dopaminergic induction.

### Example 13

### Astrocyte-Mediated DA Neuron Induction and Differentiation Was Region-Specific

Regional differences in astrocytes with respect to their factor secretion, factor response, and cytoskeletal composition have been shown in some studies (Egnaczyk et al., “Proteomic Analysis of the Reactive Phenotype of Astrocytes Following Endothelin-1 Exposur,” Proteomics 3:689-698 (2003); Lafon-Cazal et al., “Proteomic Analysis of Astrocytic Secretion in the Mouse. Comparison with the Cerebrospinal Fluid Proteome,” J Biol Chem 278:24438-24448 (2003), which are hereby incorporated by reference in their entirety). In order to determine if the induction of DA progenitors was specific to midbrain astrocytes, an hTERT expressing astrocyte line was generated from 22 wk gestational age human fetal cortical plate tissue. In parallel experiments, hES cells were co-cultured with either the midbrain astrocyte or the cortical astrocyte line as described earlier. After differentiation, no difference was found in the effect of midbrain or cortical astrocytes with respect to the proportion of total neurons generated by hES cells (FIG. 7). When astrocyte co-cultures were present in both stage 4 and 5, the percentage of βIII-tubulin expressing neurons generated from hES cells was found to be highest, at 24.9±2.2, for midbrain astrocytes and 30.1±2.8 for cortical astrocytes (FIG. 7). A significant difference was, however, found in the proportion of TH-expressing neurons generated in presence of the two astrocyte lines. As was observed with the results in the previous section, co-culturing with midbrain astrocytes throughout stages 4 and 5 resulted in the highest yield of TH⁺ neurons, at 53.7±5.5% (FIG. 7). However, with cortical astrocyte co-cultures, the percentage of TH⁺ neurons generate from hES cells was similar regardless of the presence or absence of astrocyte co-culture at stage 4 (FIG. 7). These data indicate that whereas astrocyte-mediated neuronal survival was common to both midbrain and cortical astrocytes, only midbrain astrocytes potentiated the induction of dopaminergic phenotype.

### Example 14

### Dose-dependent Effect of 6-OHDA Upon Striatal DA

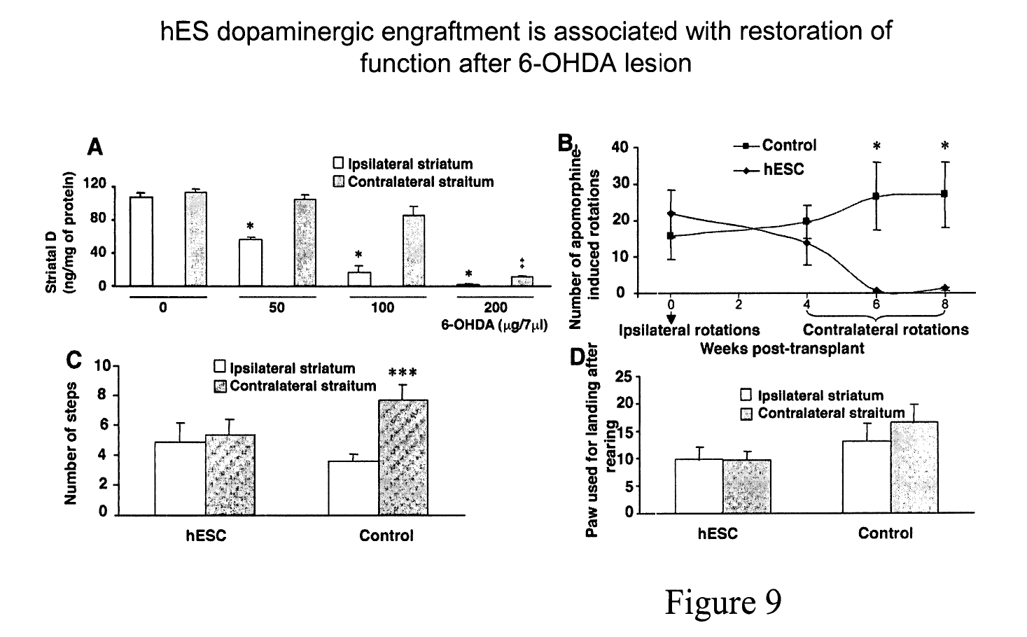

When engrafted into rat brains with chemical lesions of the nigrostriatal pathway, which vielded a Parkinson's Diseases-like syndrome, grafts of enriched dopaminergic neurons raised in co-culture with hMAST cells were able to ameliorate the motor behavior deficits of experimental Parkinson's Disease, as modeled by 6-hydroxydopamine injection (“6-OHDA”). 6-OHDA induced a reduction of DA levels in a dose-dependent manner in both the ipsilateral and contralateral striata (one-way ANOVA, p<0.001). Intrastriatal injection of 50, 100, or 200 μg /7 μl of 6-OHDA reduced the level of DA in the ipsilateral striatum by 48%, 85%, and 99%, respectively (p<0.05). Only at 200 μg/7 μl of 6-OHDA was a significant (p<0.05) 90% DA depletion observed in the contralateral striatum. The 200 μg /7 μl dose was chosen for this study, because it induced dopaminergic depletion ipsilaterally, without altering contralateral dopamine levels (FIG. 9A).

### Example 15

### Apomorphine-Induced Rotations

The transplanted rats showed a decrease in number of rotations with time (One way ANOVA, p<0.01), while the non-transplanted rats had a tendency to rotate more. Six and eight weeks after the transplantation of hESC, the number of rotations in the transplanted group differed from the number observed in the non-transplanted one (t test, p<0.05). Both groups presented ipsilateral rotations one month after the lesion, while from 2 months, the two groups showed contralateral rotations (FIG. 9B).

### Example 16

### Appendicular Dysfunction Normalized Following Transplantation

In the adjusting step test, the transplanted rats performed equally with either paw, while the non-transplanted rats used the contralateral paw more than the ipsilateral one (p<0.001). The total number of steps taken with both paws did not differ between the two groups, suggesting a similar level of motor activity in both groups (FIG. 9C). Similarly, in the cylinder test, both groups used either paw with equal frequency for landing after rearing (FIG. 9D).

### Example 17

### hES Cell Derived Progenitors Engraft and Differentiate as TH-Expressing Neurons in Striatum of 6-OHDA Lesioned Rats

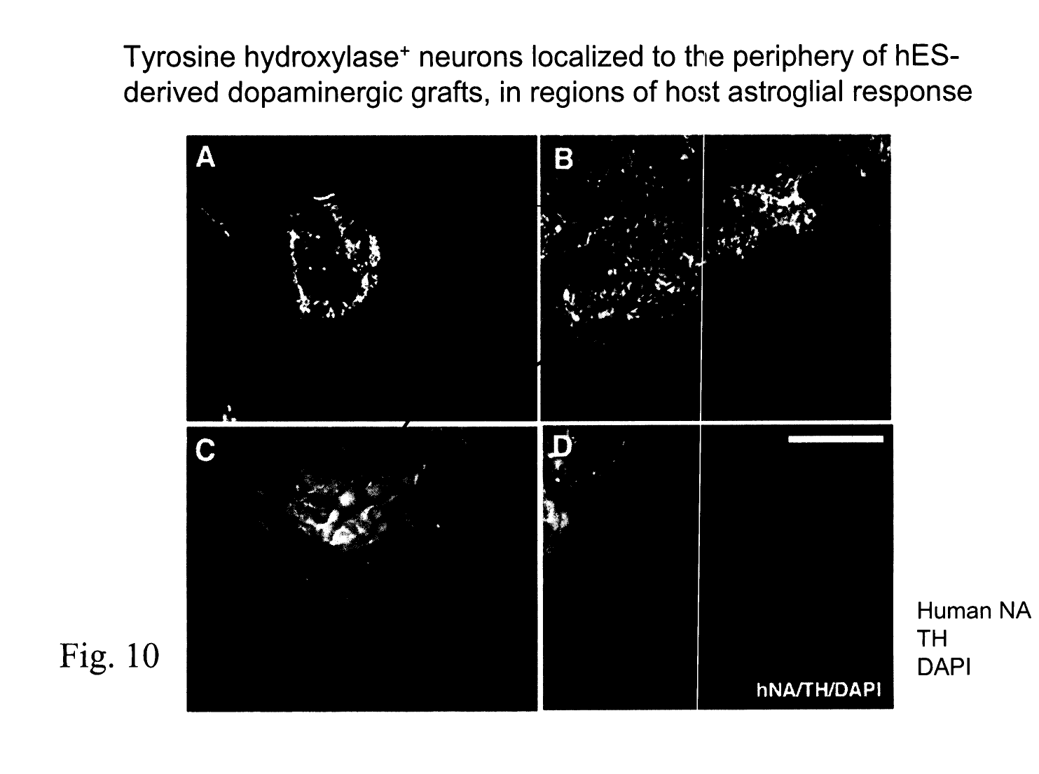

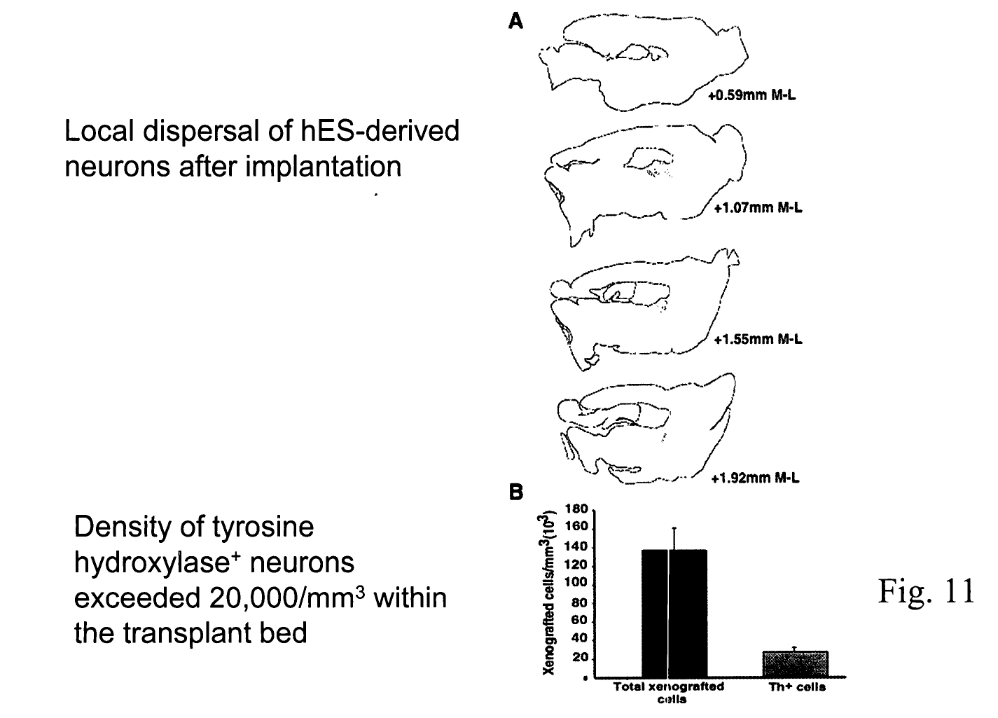

In order to relate behavioral improvement of the transplanted animals with their xenograft profile, sagittal sections of rat brains were quantified for (1) extent of media-lateral spread of xenografted cells, (2) number of cells engrafted, and (3) number of xenografted cells expressing TH. Xenografted human cells were identified by an antibody against human nuclei (hNA) (FIGS. 10A-D). Extensive engraftment of transplanted cells was seen in the striata of all 6 rats analyzed, such that the grafted cells were dispersed over an average radius of 1.6±0.6 mm (FIG. 11A). The average number of hNA+ nuclei/mm³ was 136,726±27,185 (FIG. 11B). Efficient generation of TH-expressing neurons was observed in all 6 animals. The average density of the TH⁺ cells was 27,185±4,226/mm³ with a range of 14,904/mm³-43,234/mm³ (FIG. 11B). Strikingly, a marked preferential localization of donor-derived TH⁺ cells to the donor-host interface was noted in every graft evaluated (e.g., FIGS. 10A, 12C, 13D).

### Example 18

### Dopaminergic Phenotype Persisted in Graft-Host Border Regions with Dense Local Gliosis

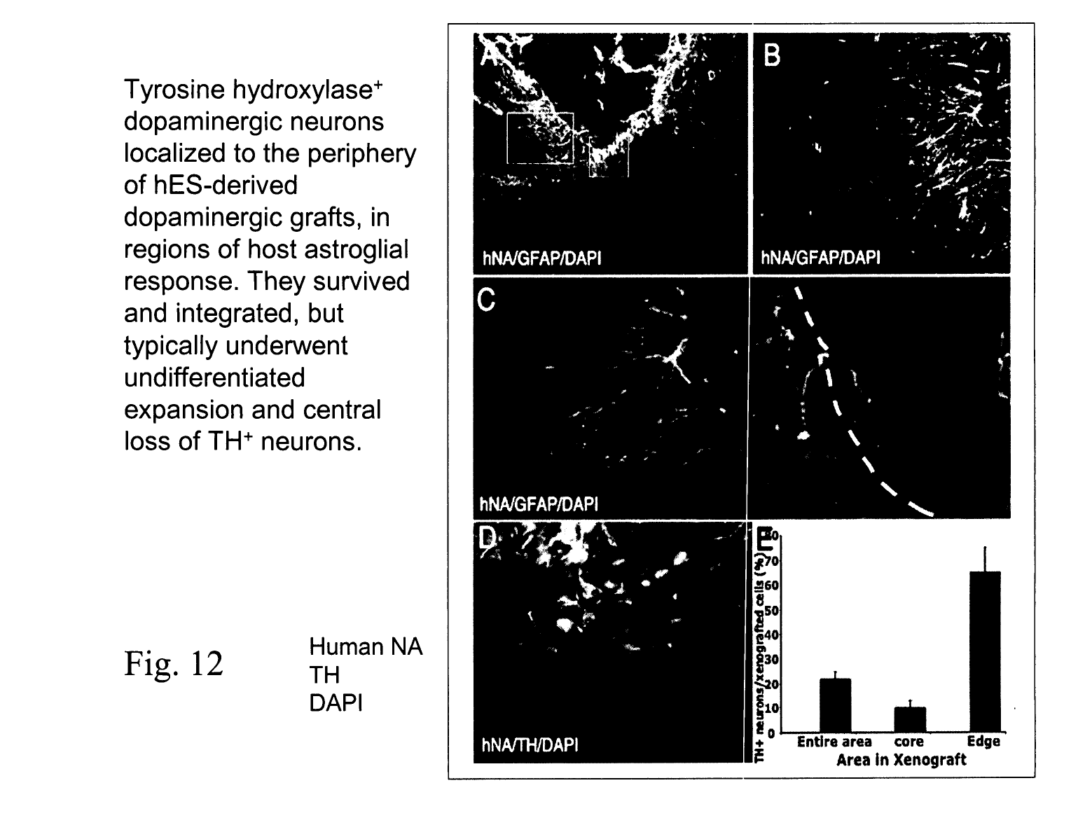

The percentage of TH⁺ neurons among the total engrafted hES cells was 21.5±3.0. In each brain, the xenograft was surrounded by host-derived reactive astrocytes (FIGS. 12A-D). To assess the significance of localization of TH⁺ neurons at edges of the xenograft area, the incidence of TH⁺ neurons in the transplant core was scored, compared to the region contiguous with the host surround. A strong correlation between the local proportion of hES cells differentiating as TH⁺ neurons and the proximity to host astrocytes, as defined by their expression of GFAP (FIG. 12E), was observed. Specifically, a significantly higher proportion of TH⁺ neurons was observed within the periphery of the graft, relative to its central core: whereas 9.8±2.9% of all cells within the central cores of the transplants were TH⁺, 65.0±10.0% of cells within the peripheral 0.2 mm of the graft expressed TH (p<0.01). As a result, even animals with a significantly lower number of engrafted cells had high numbers of TH⁺ neurons as most of the xenografted cells in these animals were in close proximity to reactive host astrocytes. This could probably explain the lack of correlation between the number of engrafted cells and the score of individual animals on the apomorphine induced rotation.

### Example 19

### Central loss of TH⁺ Cells and Undifferentiated Expansion Attended Graft Maturation

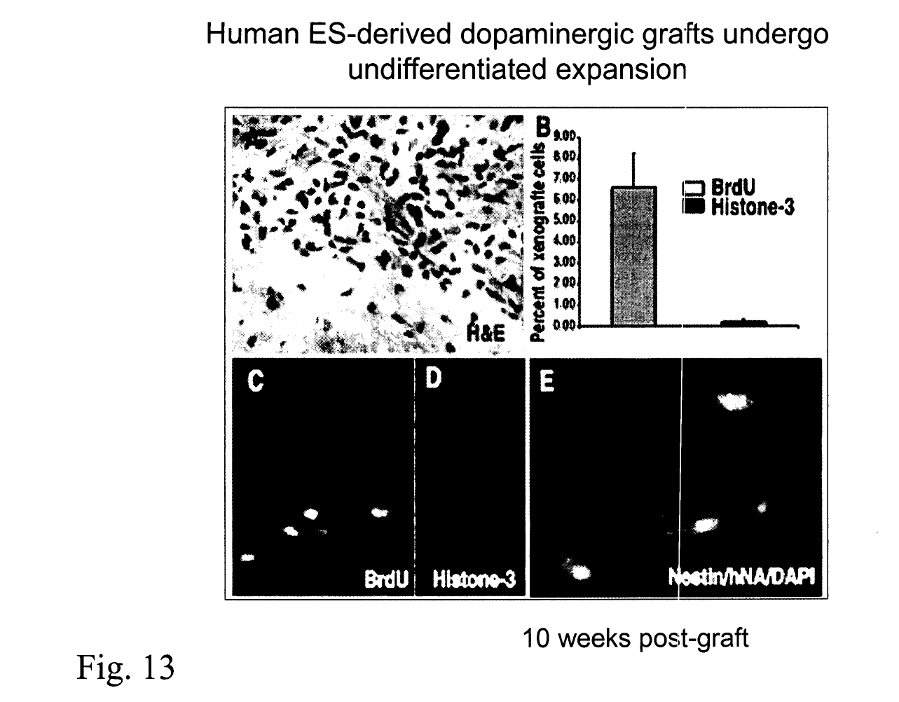

H&E stained sections of the rat brains were analyzed for the appearance of teratomas. Though, active gliosis in association with foamy macrophages, some necrosis and occasional mitotic figures were observed, no evidence of active teratoma or other tumor formation was seen in any of the sections of 3 brains sacrificed 70 days after hES cell implantation (FIG. 13A). The brains were further analyzed for mitosis among the engrafted population by immunocytochemistry for BrdU, after injecting implanted mice with BrdU 68 days after implantation (q12 hrs×3 injections, beginning 48 hrs prior to sacrifice). Among all engrafted cells in the transplant bed, 6.6±1.6% showed BrdU incorporation (FIGS. 13B-C), suggesting a daily mitotic incidence of just over 3%. Fewer cells, <1%, expressed histone3, a marker of active division (FIG. 13D). In addition, immunostaining for SSEA-4 and Oct3/4 revealed no evidence of persistent pluripotential hES cells. However, a large fraction of cells, largely localized at center of the graft, expressed nestin (FIG. 13E).

### Example 20

### A Neurogenein2 Enhancer Permits Live-Cell Identification of Ventral Neuronal Phenotypes

The large fraction of undifferentiated cells remaining and expanding within dopaminergic grafts suggested the need to purify dopaminergic neurons or their progenitors prior to transplantation. To this end, a means was developed for specifically isolating mesencephalic dopaminergic progenitor cells from human ES culture, using a mesencephalic enhancer within the neurogenin2 gene to direct expression of a GFP reporter gene in ES cells. Neurogenin-2 is a bHLH transcription factor that is expressed early in the ontogeny of many ventral neuronal phenotypes throughout the neuraxis, including spinal cord motor neurons, ventral interneurons, and the mesencephalic precursors of the substantia nigra (FIG. 12).

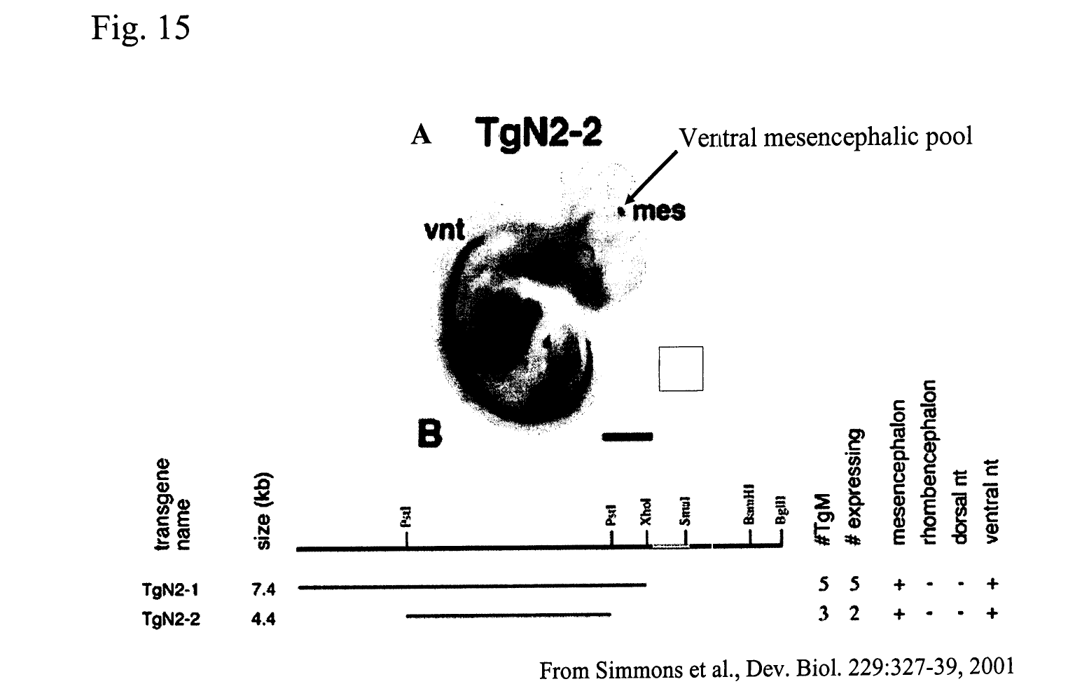

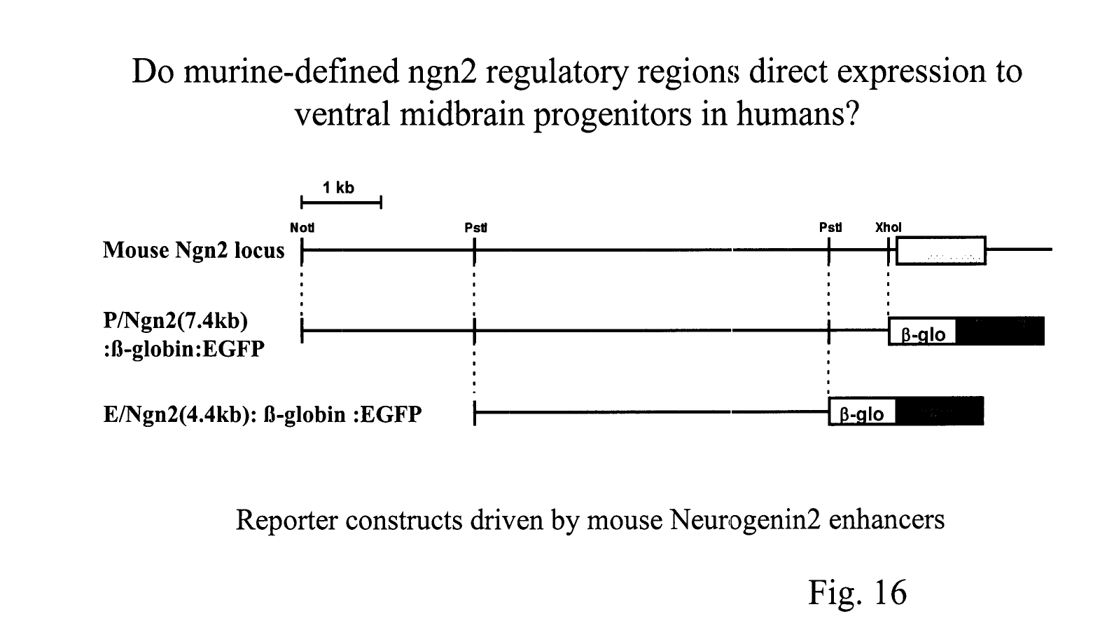

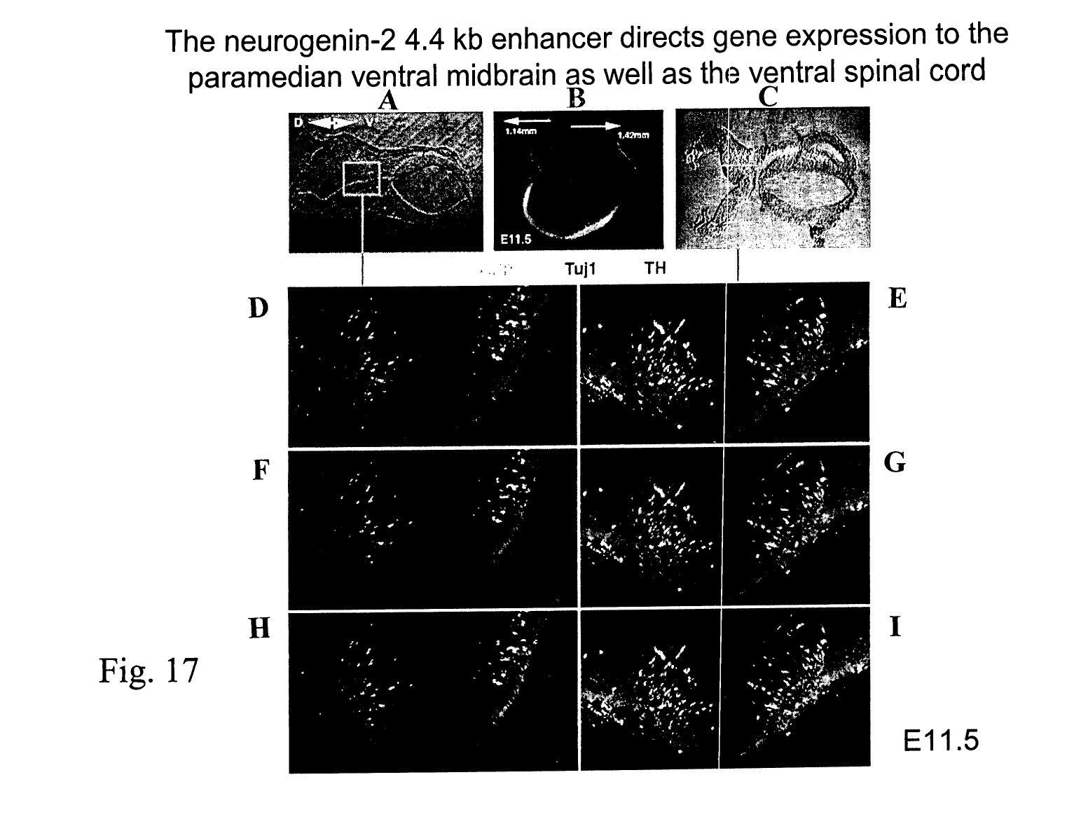

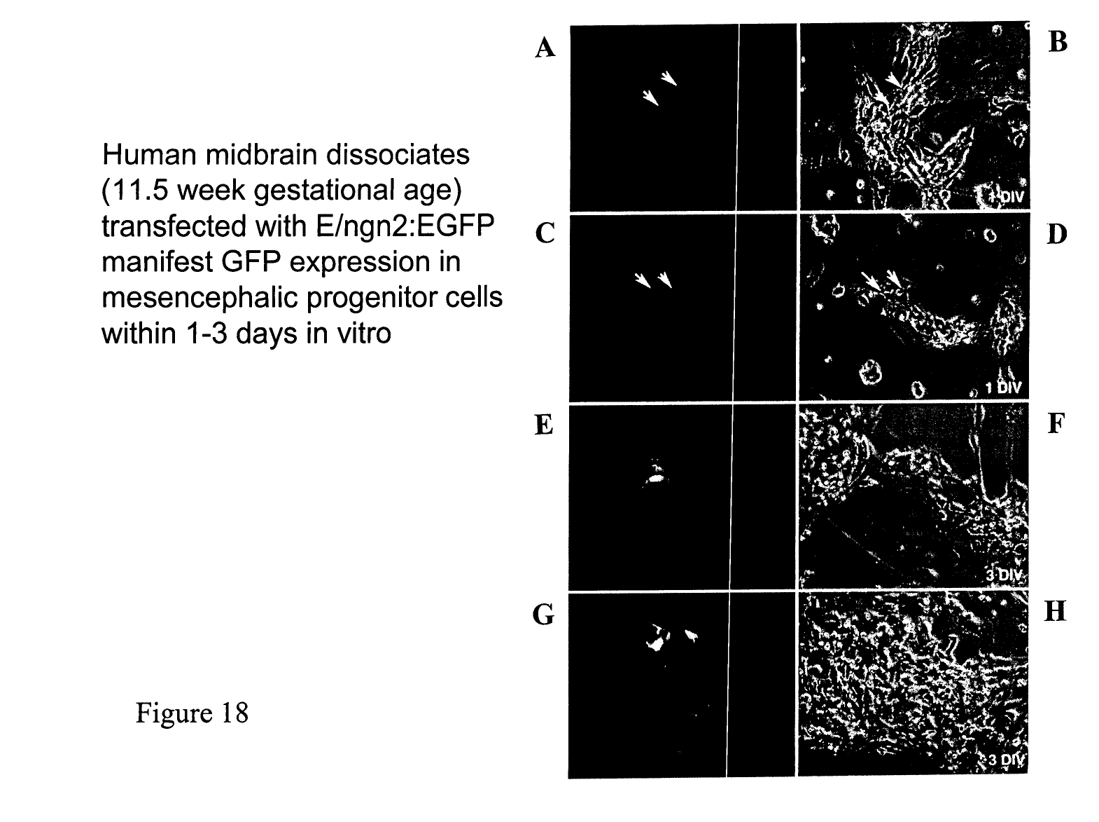

To capitalize upon the temporal and spatial pattern of neurogenin2 expression by mesencephalic progenitor cells, a selection construct of an upstream enhancer of the neurogenenin2 gene driving enhanced green fluorescence protein (EGFP) was constructed. Specifically, a 4.4 kb segment of the neurogenin2 5′ regulatory region was used that was previously shown to specify expression to both motor neurons of the spinal cord, and to substantia nigra neurons of the nigrostriatal tract (Simmons et al., “Neurogenin2 Expression in Ventral and Dorsal Spinal Neural Tube Progenitor Cells is regulated by Distinct Enhancers,” Dev Biol 229:327-339 (2001), which is hereby incorporated by reference in its entirety) (FIG. 15). By constructing a neurogenin2 enhancer-directed GFP transgenic mouse using this 4.4 kb ngn2 segment (FIG. 16), it was confirmed that this enhancer directed gene expression to ventral mesencephalic neurons, as well as to ventral spinal neurons (FIG. 17). Upon transfecting this plasmid into dissociates of fetal human midbrain, it was found that E/ngn2:EGFP indeed recognized a discrete population of neurons (FIG. 18). Within days, these cells expressed the neuronal antigens βIII-tubulin and MAP-2, and when raised in SHH/FGF8, these went on to express engrailed1, a midbrain marker, and tyrosine hydroxylase, a marker of dopamine synthesis (FIG. 18). Of note, E/ngn2:GFP-identiifed cells raised in SHH/RA instead of SHH/FGF8 largely developed as motor neurons, and expressed Islet 1 and choline acetyl-transferase, two prototypic markers of motor neuronal phenotype.

### Example 21

### FACS Isolation Based on E/neurogenin2:GFP Enriches Dopaminergic Progenitor Cells

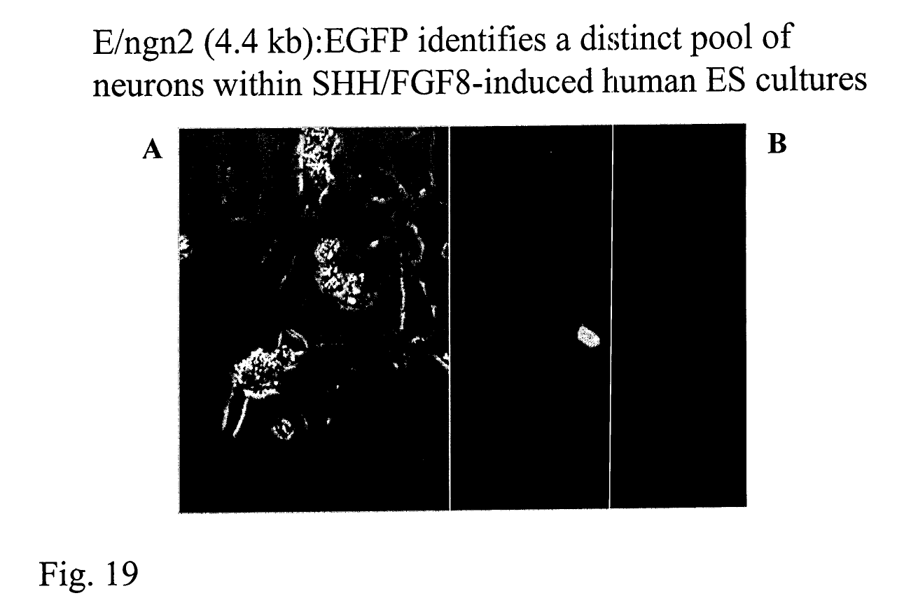

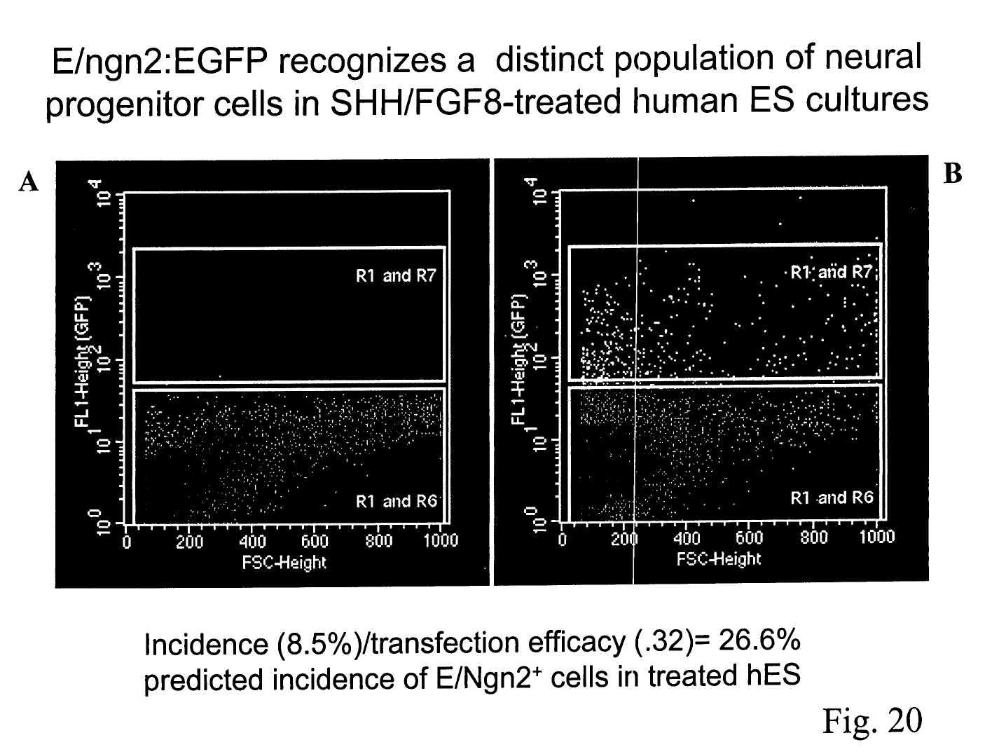

E/neuroigenin2-driven GFP expression selected a population of cells from both fetal midbrain (FIG. 18) and human ES culture (FIG. 19). On this basis, fluorescence activated cell sorting was used to isolate the E/ngn2:EGFP⁺ population (FIG. 20). These cells developed tyrosine hydroxylase expression as dopaminergic neurons, indicating the specificity with which dopaminergic neuronal progenitors may be extracted from both mixed hES cultures and primary dissociates of fetal human forebrain. The procedure by which this high-grade enrichment—to virtual purity—of human mesencephalic dopaminergic neural progenitor cells was accomplished is schematized in FIG. 1.

In this study, it was asked if the generation of phenotypically-restricted neurons from human ES cells could be potentiated by early exposure to fetal mesencephalic human glia to better replicate the in vivo environment of the fetal mesencephalon. To address this issue, a telomerase-immortalized line of human mesencephalic astrocytes, derived from second trimester fetal ventral midbrain, was generated. When coupled with previously established protocols for accentuating dopaminergic differentiation, mesencephalic glial co-culture indeed strongly potentiated dopaminergic neuronal differentiation from human ES cells. When embryoid bodies were generated in mesencephalic glial co-culture with concurrent FGF8/SHH induction, tyrosine hydroxylase-expressing neurons were by the far the major neuronal phenotype produced, in comparison >60% of all neurons by 4 weeks. This higher efficiency method of generating dopaminergic neurons from hES cells through mesencephalic astrocytic co-culture, yielded substantial enrichment of dopaminergic neurons and their progenitors. The utility of these enriched dopaminergic cells as graft vehicles was assessed, by transplanting hES-derived dopaminergic progenitors to 6-hydroxydopamine lesioned rat striata. Excellent graft acceptance and survival, and the significant improvement of motor performance after 6-OHDA lesion, relative to untreated controls, was observed.

It is important to note that by using human astrocytic feeders and serum-free culture, dopaminergic neurons under largely humanized conditions were generated and propagated. Specifically, by using H9 cells raised under feeder-free conditions, and hMAST-TERT mesencephalic astrocytes rather than mouse embryonic fibroblasts as the source of conditioned media, the generation and purification of human dopaminergic neurons was achieved with no specific exposure to animal cells or products.

Together, these data indicate that co-culture of human ES cells with telomerase-immortalized human mesencephalic astrocytes during and after the period of SHH/FGF8-induced neuronal induction strongly potentiates dopaminergic neurogenesis from human ES cells and, that upon striatal implantation into 6-OHDA-lesioned hosts, these cells engraft, generate dopaminergic neurons, and ameliorate the behavioral deficits of striatal dopaminergic denervation. However, it is also noted that the incidence of neurons manifesting dopaminergic antigenicity fell sharply over a month in vivo; this was especially remarkable in light of the highly enriched nature of these cells at the time of implantation. In addition, undifferentiated ES cell expansion within several of the grafts was noted. These late complications of otherwise successful hES-derived dopaminergic neuronal grafts indicate that the need not only to establish strategies for dopaminergic neurogenesis and enrichment, but also methods for ridding these preparations from persistent undifferentiated cells, while sustaining the mature phenotype of those dopaminergic neurons that do successfully engraft.

To address this latter need, a neurogenin2 enhancer (E/ngn2)-driven, GFP-based strategy was established for separating dopaminergic progenitors, allowing their high-yield enrichment to virtual purity. The specificity of the ventral mesencephalic ngn2 enhancer element and its selection of ventral mesencephalic derivatives was validated, by generating an E/ngn2:βglobin:EGFP transgenic mouse. This confirmed the ventral mesencephalic expression of the E/ngn2 enhancer that was then used for somatic selection. By serially combining SHH/FGF8/hMAST-potentiated induction of dopaminergic neurogenesis with FACS selection of the dopaminergic neurons after E/ngn2:GFP transfection, the high-efficiency isolation of human dopaminergic neurons was achieved. This higher efficiency method of generating dopaminergic neurons from hES cells through mesencephalic astrocytic co-culture, coupled with the E/ngn2-based selection that was developed for this work, yielded hitherto unachieved enrichment of dopaminergic progenitors.

An additional embodiment of this method of preparing highly enriched populations of dopaminergic progenitor cells is to combine it with hTERT (human telomeric extension reverse transcriptase)-transduction to achieve telomerase over-expression; the resultant telomerase-expressing dopaminergic progenitor lines would be capable of prolonged expansion as phenotypically-restricted ventral mesencephalic neuronal progenitors (e.g. Roy et al., “Telomerase Immortalization of Neuronally Restricted Progenitor Cells Derived from the Human Fetal Spinal Cord,” Nature Biotechynology 22:297-30 (2004), which is hereby incorporated by reference in its entirety). Most if not all of these cells would be expected to mature as dopaminergic neurons.

An additional advantage of this method is that it has allowed human dopaminergic neurons to be generated in entirely humanized conditions. By using H9 cells raised under feeder-free conditions and using mesencephalic astrocytes rather than mouse fibroblasts as the source of conditioned media, the generation and purification of human dopaminergic neurons in entirely humanized conditions was achieved, with no exposure to animal cells or products. By avoiding exposure to animal cells or products thereof, the cross-species immunogenicity to which ES cells are prone may be avoided, allowing their use in human allograft.

Although preferred embodiments have been depicted and described in detail herein, it will be apparent to those skilled in the relevant art that various modifications, additions, substitutions, and the like can be made without departing from the spirit of the invention and these are therefore considered to be within the scope of the invention as defined in the claims which follow.

1. A method of isolating an enriched or purified population of dopaminergic neuronal progenitor cells from a population of embryonic stem cells comprising:
providing a population of embryonic stem cells;
inducing production of dopaminergic neuronal progenitor cells from the population of embryonic stem cells;
selecting a promoter or enhancer which functions only in dopaminergic neuronal progenitor cells;
introducing a nucleic acid molecule encoding a marker protein under control of said promoter or enhancer into the induced population of embryonic stem cells;
allowing the dopaminergic neuronal progenitor cells to express the marker protein; and
separating the cells expressing the marker protein from the induced population of embryonic stem cells, whereby an enriched or purified population of dopaminergic neuronal progenitor cells is isolated.

2. The method of claim 1, wherein said introducing comprises viral mediated transduction of the induced population of embryonic stem cells.

3. The method of claim 2, wherein said viral mediated transduction comprises adenovirus-mediated transduction, retrovirus-mediated transduction, lentivirus-mediated transduction, or adeno-associated virus-mediated transduction.

4. The method of claim 1, wherein said introducing comprises electroporation.

5. The method of claim 1, wherein said introducing comprises biolistic transformation.

6. The method of claim 1, wherein said introducing comprises liposomal mediated transformation.

7. The method of claim 1, wherein the marker protein is a fluorescent protein and said separating comprises fluorescence activated cell sorting.

8. The method of claim 1, wherein the marker protein is either lacZ/beta-galactosidase or alkaline phosphatase.

9. The method of claim 1, wherein said promoter or enhancer is for neurogenin-2.

10. The method of claim 1, wherein said promoter or enhancer is a promoter or enhancer for genes in the dopamine synthesis pathway, a promoter or enhancer for dopamine transport proteins, or a promoter or enhancer for genes expressed differentially in the ventral midbrain.

11. The method of claim 1, wherein the population of embryonic stem cells is in a cell culture.

12. The method of claim 11, wherein the cell culture further comprises astrocytes.

13. The method of claim 12, wherein the astrocytes are human mid-brain astrocytes.

14. The method of claim 12, wherein the astrocytes are immortalized.

15. The method of claim 1, wherein the embryonic stem cells are of human origin.

16. The method of claim 1 further comprising:
transplanting the separated cells into a subject.

17. The method of claim 16, wherein the subject has a dopamine-depletion disease.

18. The method of claim 17, dopamine-depletion disease is Parkinson's Disease.

19. The method of claim 1, wherein said inducing is carried out by administering an inducer selected from the group consisting of FGF2, FGF8, FGF20, SHH, and mixtures thereof.

20. The method of claim 1 further comprising:
differentiating the enriched or purified population of dopaminergic neuronal progenitor cells into dopamineric neurons.

21. An enriched or purified population of dopamineric neurons produced by the method of claim 20.

22. The enriched or purified population of dopaminergic neuron according to claim 21, wherein the dopaminergic neurons are of human origin.

23. An enriched or purified population of dopaminergic neuronal progenitor cells produced by the method of claim 1.

24. The enriched or purified population of dopaminergic neuronal progenitor cells according to claim 23, wherein the dopaminergic neuronal progenitor cells are of human origin.

25. A method of producing an enriched or purified population of dopaminergic neuronal progenitor cells from a population of embryonic stem cells comprising:
selecting a promoter or enhancer which functions only in said dopaminergic neuronal progenitor cells;
introducing a nucleic acid molecule encoding a marker protein under control of said promoter or enhancer into the population of human embryonic stem cells;
inducing the population of embryonic stem cells to produce a mixed population of cells comprising dopaminergic neuronal progenitor cells;
allowing the dopaminergic neuronal progenitor cells to express the marker protein; and
separating the cells expressing the marker protein from the mixed population of cells, whereby an enriched or purified population of dopaminergic neuronal progenitor cells is isolated.

26. The method of claim 25, wherein said introducing comprises viral mediated transduction of the population of embryonic stem cells.

27. The method of claim 26, wherein said viral mediated transduction comprises adenovirus-mediated transduction, retrovirus-mediated transduction, lentivirus-mediated transduction, or adeno-associated virus-mediated transduction.

28. The method of claim 25, wherein said introducing comprises electroporation.

29. The method of claim 25, wherein said introducing comprises biolistic transformation.

30. The method of claim 25, wherein said introducing comprises liposomal mediated transformation.

31. The method of claim 25, wherein the marker protein is a fluorescent protein and said separating comprises fluorescence activated cell sorting.

32. The method of claim 25, wherein the marker protein is either lacZ/beta-galactosidase or alkaline phosphatase.

33. The method of claim 25, wherein said promoter or enhancer is for neurogenin-2.

34. The method of claim 25, wherein said promoter or enhancer is a promoter or enhancer for genes in the dopamine synthesis pathway, a promoter or enhancer for dopamine transport proteins, or a promoter or enhancer for genes expressed differentially in the ventral midbrain.

35. The method of claim 25, wherein the population of human embryonic stem cells is in a cell culture.

36. The method of claim 35, wherein the cell culture further comprises astrocytes.

37. The method of claim 36, wherein the astrocytes are human mid-brain astrocytes

38. The method of claim 36, wherein the astrocytes are immortalized.

39. The method of claim 25, wherein the embryonic stem cells are of human origin.

40. The method of claim 25 further comprising:
transplanting the separated cells into a subject.

41. The method of claim 40, wherein the subject has a dopamine-depletion disease.

42. The method of claim 41, wherein the dopamine-depletion disease is Parkinson's Disease.

43. The method of claim 25, wherein said inducing is carried out by administering an inducer selected from the group consisting of FGF2, FGF8, FGF20, SHH, and mixtures thereof.

44. The method of claim 25 further comprising:
differentiating the enriched or purified population of dopaminergic neuronal progenitor cells into dopamineric neurons.

45. An enriched or purified population of dopaminergic neurons produced by the method of claim 44.

46. The enriched or purified population of dopaninergic neurons of claim 41, wherein the dopaminergic neurons are of human origin.

47. An enriched or purified population of dopaminergic neuronal progenitor cells produced by the method of claim 25.

48. The enriched or purified population of dopaminergic neuronal progenitor cells of claim 47, wherein the dopaminergic neurons are of human origin.

49. An enriched or purified preparation of isolated dopaminergic neurons derived from embryonic stem cells.

50. The enriched or purified preparation of isolated dopaminergic neurons of claim 49, wherein the dopaminergic neurons are of human origin.

51. The enriched or purified preparation of isolated dopaminergic neurons of claim 50, wherein a neurogenin-2 promoter or enhancer functions in all cells of the enriched or purified preparation.

52. The enriched or purified preparation of dopaminergic neurons of claim 49, wherein the enriched or purified preparation of dopaminergic neurons are generated from the embryonic stem cells without contacting non-human cells.

53. The enriched or purified preparation of dopaminergic neurons of claim 49, wherein the preparation comprises at least 90% of the isolated dopaminergic neurons.

54. The enriched or purified preparation of dopaminergic neurons of claim 49, wherein the preparation comprises at least 99% of the isolated dopaminergic neurons.

55. An enriched or purified preparation of isolated dopaminergic neuronal progenitor cells derived from embryonic stem cells.

56. The enriched or purified preparation of isolated dopaminergic neuronal progenitor cells of claim 55, wherein the dopaminergic neuronal progenitor cells are of human origin.

57. The enriched or purified preparation of isolated dopaminergic neuronal progenitor cells of claim 55, wherein a neurogenin-2 promoter or enhancer functions in all cells of the enriched or purified preparation.

58. The enriched or purified preparation of isolated dopaminergic neuronal progenitor cells of claim 55, wherein the enriched or purified preparation of dopaminergic neuronal progenitor cells are generated from embryonic stem cells without contacting non-human cells.

59. The enriched or purified preparation of isolated dopaminergic neuronal progenitor cells of claim 55, wherein the preparation comprises at least 90% of the isolated dopaminergic neuronal progenitor cells.

60. The enriched or purified preparation of isolated dopaminergic neuronal progenitor cells of claim 55, wherein the preparation comprises at least 99% of the isolated dopaminergic neuronal progenitor cells.

61. A cell line of immortalized human mid-brain astrocytes.

62. The cell line of claim 61, wherein the astrocytes are fetal-derived.

63. The cell line of claim 61, wherein the astrocytes are adult-derived.

64. The cell line of claim 61, wherein the astrocytes are TERT-immortalized.
